(19) United States
(12) Patent Application Publication
(10) Pub. No.: US 2008/0213350 A1
(43) Pub. Date: Sep. 4, 2008

(54) Encapsulation of nucleic acids in liposomes

(75) Inventors: Young Tag Ko, Boston, MA (US); Ulrich Bickel, Amarillo, TX (US)

Correspondence Address: JACKSON WALKER LLP, 901 MAIN STREET, SUITE 6000, DALLAS, TX, 75202-3797, US

(73) Assignee: Texas Tech University System, Lubbock, TX (US)

(21) Appl. No.: 12/070,602

(22) Filed: Feb. 20, 2008

Related U.S. Application Data
(60) Provisional application No. 60/902,277, filed on Feb. 20, 2007.

(51) Int. Cl. A61K 9/127

(52) U.S. Cl. 424/450

(57) ABSTRACT

Complexes of nucleic acid and cationic polymer, which are encapsulated in liposomes for the purpose of delivering nucleic acid and methods for producing encapsulated complexes.

### RELATED APPLICATIONS

This application claims priority to U.S. Provisional Patent Application Ser. No. 60/902,277, entitled “ENCAPSULATION OF NUCLEIC ACIDS IN LIPOSOMES” filed on Feb. 20, 2007, having Young Tag Ko and Ulrich Bickel, listed as the inventor(s), the entire content of which is hereby incorporated by reference.

### STATEMENT OF RIGHTS TO INVENTIONS MADE UNDER FEDERALLY SPONSORED RESEARCH

This invention was made in part during work supported by a grant from the National Institutes of Health (NIH grant #5R01NSO45043-04). The government may have certain rights in the invention.

### BACKGROUND

The current invention relates to complexes of nucleic acid and cationic polymer, which are encapsulated in liposomes for the purpose of delivering DNA to a tissue within an organism.

### PEG-Stabilized Nanoparticulate Drug Delivery Systems

The aim of any drug delivery system is to modulate the pharmacokinetics and/or tissue distribution of the drug in beneficial ways, i.e., prolong blood circulation time or enhance target tissue delivery. Incorporating an existing therapeutic agent into a new delivery system can significantly improve its performance in terms of efficacy, safety, and patient compliance. Development of delivery systems for biopharmaceuticals such as proteins, peptides, carbohydrates, nucleotides has been an enormous challenge because these biopharmaceuticals are often large molecules that are subject to rapid degradation by enzymes, short blood circulation time, rapid clearance and immunogenicity in the blood stream. Moreover, they have a limited ability to cross cell membranes and generally cannot be delivered orally.

Among the variety of delivery systems that have been devised to improve the clinical properties of biopharmaceuticals over the years are many particulate colloidal carrier systems such as liposomes, nanoparticles, microemulsions, micellar systems. Although sometimes successful, the particulate delivery systems still have a number of limitations. The particulate delivery systems are rapidly cleared from blood and sequestered into liver and spleen as part of reticuloendothelial system (RES)(Szebeni 1998). Upon intravenous injection, the particulate carrier systems are rapidly cleared from the blood by macrophages of the RES. The rapid sequestration of intravenously injected colloidal particles from the blood by the RES is problematic for efficient targeting of therapeutic agents to target sites other than macrophages of the reticuloendothelial organs. As a result, there has been a growing interest in the engineering of colloidal carrier systems that upon intravenous administration are capable of avoiding rapid recognition by the RES and thus remain in the blood circulation for a long period.

One of the ways to escape RES recognition and thus provide long circulating properties has been surface modification of the particulate systems in a way that confers a steric barrier against interactions with blood components, which are responsible for RES uptake and rapid clearance. Among the molecules which have been explored for the surface modification are polysaccharides and glycoproteins in an attempt to exploit the surface strategies of some microorganisms to avoid immune recognition. Synthetic polymers have also been exploited for this purpose.

The majority of these synthetic materials are based on polyethylene glycol (PEG) and its derivatives. PEG is a linear addition polymer of ethylene oxide and water. PEG exhibits a low degree of immunogenicity and has been approved for a wide range of biomedical applications including injectable, topical, rectal and nasal formulations. The polymer backbone of PEG is essentially inert in a biological environment and in most chemical reaction conditions. The terminal primary hydroxyl groups are available for the formation of a number of derivatives. Since PEG attachment to bovine catalase was first developed (Abuchowski, McCoy et al. 1977), the conjugation of various molecules, especially therapeutic proteins and peptides, with PEG (pegylation) have been used to enhance the delivery of the therapeutic molecules by modifying pharmacokinetics and pharmacodynamics (Harris, Martin et al. 2001). Pegylation alters the immunological, pharmacokinetic and pharmacodynamic properties of the therapeutic proteins in ways that can extend its potential uses. Pegylation also changes physicochemical properties of the protein molecules such as conformation, steric hindrance, electrostatic binding properties, hydrophobicity, local lysine basicity. These changes reduce systemic clearance of the proteins by decreasing renal clearance, proteolysis, opsonization and thus RES uptake.

Although PEG conjugates of therapeutic proteins and peptides have generated the most interest and have been the main targets for pegylation (Petersen, Fechner et al. 2002), a variety of molecules such as small molecule drugs, lipids, genetic materials, and biological polymers can be conjugated to PEG. This, in turn, has launched a whole new range of better drug delivery systems with enhanced properties. PEG conjugation has been tried and found applicable with particulate colloidal drug carrier systems such as liposomes, nanoparticles, polymer micelles, and microemulsions in an attempt to improve their in vivo behavior upon intravenous administration.

### PEGylation of Liposomes

Liposomes are spherical lipid bilayer vesicles with an aqueous core compartment. Liposomes are formed by self-assembly of phospholipid molecules in an aqueous environment. The amphiphilic phospholipid molecules form a closed bilayer sphere in aqueous medium to shield their hydrophobic groups from the aqueous environment and maintain contact with the aqueous phase via the hydrophilic head groups. The closed sphere of the phospholipid bilayer can encapsulate aqueous soluble drugs within the central aqueous compartment or lipid soluble drugs within the bilayer membrane. The encapsulation of drugs within liposomes alters pharmacokinetics and biodistribution of the drugs, and thus liposomes can be exploited as a drug delivery system. Liposomes have been widely used as a drug carrier for the improved delivery of a variety of drugs such as chemotherapeutic agents, imaging agents, antigens, genetic materials, and immunomodulators. In the majority of cases, liposomal systems provide less toxicity and better efficacy than the free active ingredients.

Conventional liposomes are typically composed of only phospholipids and/or cholesterol. These are characterized by a relatively short blood circulation time. When administered in vivo by a variety of parenteral routes, they show a strong tendency to accumulate rapidly in the phagocytic cells of the mononuclear phagocyte system (MPS). To overcome this problem, long-circulating liposomes or sterically stabilized liposomes (SSL) have been developed. At present the most popular way to produce long circulating liposomes is to attach hydrophilic polymer polyethylene glycol (PEG) covalently to the outer surface of the liposomes. Such PEG coating of the liposomes provides prolonged circulation time by creating a steric barrier against interactions with blood components and cellular membranes.

For incorporation of PEG into the liposomal bilayer, a number of PEG-conjugated lipids have been prepared using phospholipids that contain a primary amino group such as phosphatidylethanolamine (PE) (Blume, Cevc et al. 1993), a carboxyl group (Allen, Hansen et al. 1991), an epoxy group (Papahadjopoulos, Allen et al. 1991) or a diacylglycerol moiety (Mori, Klibanov et al. 1991). It is known that the PEG conjugation has no significant influence on the liposome forming ability of the conjugates. Alternatively, activated PEG can be anchored to reactive phospholipid groups of preformed liposomes (Senior, Delgado et al. 1991). Another strategy has utilized the transfer of PEG-phospholipid conjugates from the micellar phase into the lipid bilayer of preformed vesicles (Uster, Allen et al. 1996). To date, PEG-grafted liposomes with the size range of 70 to 200 nm and 3 to 7 mol % PEG2000-DSPE or DPPE in addition to various amounts of phospholipids and cholesterol are the best engineered long-circulating liposomes, typically showing a circulation half-life of 12-20 hours in rats and mice and 40-60 hours in human (Woodle 1998).

The effect of PEG molecular weight on prolonging the circulation time of the PEG-grafted liposomes was studied in mice using DSPE-PEG conjugates from PEG1000, 2000, 5000, 12000, resulting in extended circulation times by DSPE-PEGs with molecular weight of 1000 and 2000 more than other DSPE-PEGs with higher molecular weight of 5000 and 12000 (Kakudo, Chaki et al. 2004). This is different from other systems, where an increase in molecular weights of PEG results in an increase in steric stabilization effects. The decrease in steric stabilization with the increased molecular weight of PEG chains can be explained by intermembrane transfer of the PEG-phospholipid conjugates, thus loss of the lipid derivatives from the liposomal surface. The intermembrane transfer of the PEG-phospholipids conjugates is expected to take place earlier in the PEG-phospholipid conjugates with higher molecular weight PEG and decrease with increasing fatty acid chain length (Silvius and Zuckermann 1993). This suggests that increasing the molecular weight of PEG chains leads to loss of PEG-lipids from the vesicles. In addition, the increasing molecular weight of PEG chain increases PEG chain-chain interaction which may lead to phase separation in the liposome with large chain PEG-PE, thus poor steric protection of the liposomes (Bedu-Addo, Tang et al. 1996).

The incorporation of cholesterol into the liposomal bilayer can further improve surface protection by PEG coating (Bedu-Addo, Tang et al. 1996). Upon incorporation of high concentration of cholesterol (>30 mol %) into the lipid bilayer containing PEG (12,000)-DPPE, the formation of phase separated lamellae, which otherwise occurred at all concentrations of PEG-PE conjugate due to PEG chain-chain interaction, was completely inhibited. This was due to an increase in the bilayer cohesive strength and hence a reduction in the formation of phase separated lamellae. Because of their relatively inflexible structures, cholesterols act as a spacer keeping lipid chains apart and reducing PEG chain-chain interactions. At higher concentrations of PEG-PE, solubilization of the bilayer occurs with preferential solubilization of cholesterol over phospholipids. Even in the presence of cholesterol the steric protection of long chain PEG-PE is relatively poor. This is presumably due to the reduction in the intramolecular expansion with increase in molecular weight of PEG chains. The reduced intramolecular expansion can lead to coil shrinkage and hence reduced chain flexibility. For these reasons the most suitable formulations for prolonged circulation time contains more than 30 mol % cholesterol and equal or less than 7 mol % short PEG-PE.

Size of pegylated liposomes also affects the blood circulation time and biodistribution. The effect of liposome size on circulation time and biodistribution has been studied with three different sizes (d>300 nm, 150-200 nm, <70 nm) of liposomes containing PEG-PE conjugates (Litzinger, Buiting et al. 1994; Harashima, Hiraiwa et al. 1995). The liposomes of intermediate size showed the longest circulation time, whereas the large and small liposomes accumulated to elevated levels in spleen and liver. Since liposomes accumulated in liver were localized to Kupffer cells, not to parenchymal cells, the high level of accumulation of small liposomes in liver doesn't seem to be due to extravasation through the fenestrated liver endothelium, which are 100 to 150 nm in diameter (Braet, De Zanger et al. 1995). Instead, size dependence of steric barrier activity shown by a serum protein binding assay where small liposomes showed increased protein binding may be the reason for reduced circulation times of the small liposomes. This decreased steric barrier of the small liposomes may result in increased susceptibility to opsonization and thus more rapid clearance from the circulation. The large liposomes accumulated in spleen were localized in the red pulp and marginal zone, indicating that uptake of the large liposomes in spleen may occur by means of a filtration mechanism through reticular meshwork with the slit size of 200 to 500 nm in width (Moghimi, Porter et al. 1991).

The administered dose of the liposomes can also affect pharmacokinetics. The pharmacokinetics of pegylated liposomes as a function of dose was investigated in comparison to conventional liposomes (Allen and Hansen 1991). Clearance of the conventional liposomes showed marked dose dependence with RES uptake decreasing and percentage of indicated dose (% ID) in blood increasing as dose increased, indicating a saturation of RES. On the other hand, the plasma half-life of the pegylated liposomes containing DSPE-PEG1990 remained relatively unchanged and the plasma AUC increased linearly as dose of the liposomes increased, suggesting dose-independent first-order kinetics. The pharmacokinetic behavior and biodistribution of the pegylated liposomes can also be affected by repeated intravenous administration. The circulation half-life of the second injection of the pegylated liposomes was dramatically decreased and biodistribution 4 hours after the second dose showed a significantly reduced blood content accompanied by a highly increased uptake in the liver and spleen (Laverman, Boerman et al. 2001). The enhanced clearance effect of the pegylated liposomes upon repeated administration seems to be caused by a soluble serum factor and mediated by RES since the depletion of hepatosplenic macrophages abolished the enhanced clearance effect.

A wide array of anticancer drugs has been encapsulated within pegylated liposomes in an effort to target such agents to tumors. Pegylated liposomes with about 100 nm in size can passively target solid tumors by extravasation into their extracellular space upon intravenous administration as a result of the discontinuous leaky microvasculature in tumors. Doxorubicin, an amphiphilic anticancer agent, has been a most extensively studied drug for the liposomal formulation. Incorporation of doxorubicin into pegylated liposomes composed of 1,2-Distearoyl-sn-Glycero-3-Phosphocholine/Cholesterol/1,2-Distearoyl-sn-Glycero-3-Phosphoetnanolamine-N-[Amino(Polyethylene glycol)2000 (HSPC/Cho1/PEG2000-DSPE (56:39:5)) altered the pharmacokinetics of the drug. Compared with conventional liposomal formulations, pegylated liposomal doxorubicin showed less RES uptake and reduced leakage of the drugs from vesicles during circulation. The pharmacokinetics of pegylated liposomal doxorubicin are characterized by a smaller volume of distribution, slower plasma clearance, and extremely long circulation half-life compared to conventional liposomal doxorubicin or free doxorubicin. The long circulation time and ability of pegylated liposomes to extravasate through leaky tumor vasculature results in enhanced accumulation of doxorubicin within tumor tissue and thus better antitumor activity than equivalent doses of conventional liposome encapsulated doxorubicin or free doxorubicin. Low peak plasma concentrations of free doxorubicin after administration of pegylated liposome encapsulated doxorubicin and the reduced tendency of the liposomal drug to accumulate in myocardium suggest a reduction in cardiac toxicity (Coukell and Spencer 1997; Gabizon and Martin 1997). Indeed, pegylated liposomal formulation of doxorubicin was approved in 1995 for the treatment of Kaposi's sarcoma and is under clinical trial for metastatic ovarian cancer.

In order to further enhance selective delivery of pegylated liposomes, active targeting of the PEG-grafted long-circulating liposomes may be achieved by conjugating targeting moiety such as antibodies or ligands for specific receptors to the surface of liposomes or to the distal ends of PEG chains to produce stealth immunoliposomes (SIL). The effectiveness of the antibody attached on the surface of liposomes in targeting the liposome is dependent on the density and molecular weight of PEG on the liposome surface, since a high density and high molecular weight of PEG reduce not only the RES uptake, but also the immunospecific antigen-antibody binding by shielding the antibody from the antigens. However, antibody attached to the PEG terminal of the pegylated liposomes is not sterically hindered and thus the exposure of antibodies to the target is enhanced by their attachment to the distal ends of the PEG chains while free PEG is effective in increasing the blood concentration of immunoliposomes by enabling them to evade RES uptake (Kakudo, Chaki et al. 2004). The ability to selectively target liposomal anticancer drugs such as doxorubicin via specific antibodies against antigens expressed on malignant cells could improve the therapeutic effectiveness of the liposomal preparations as well as reduce adverse side effects associated with chemotherapy. The specific binding, in vitro cytotoxicity, and in vivo antineoplastic activity of doxorubicin encapsulated in stealth immunoliposomes (SILs) coupled to monoclonal Ab anti-CD19 were investigated against malignant B lymphoma cells expressing CD19 surface antigen. The results showed 3-fold increased binding and higher toxicity of the SILs with a human CD19+ B lymphoma cells in comparison with non-targeted stealth liposomes and significantly increased effectiveness in immunodeficient mice (Lopes de Menezes, Pilarski et al. 1998).

In summary, pegylation has been a standard method for improving pharmacokinetics, pharmacodynamics and clinical effects of various therapeutic biopharmaceuticals such as proteins and peptides, leading to some successful results with FDA approval. These include pegylated interferon-α for the treatment of chronic hepatitis C virus infection, pegylated human granulocyte colony-stimulating factor (G-CSF) (Neulasta) for the treatment of different types of tumors or related clinical problems, and pegylated insulin-like growth factor-1 (IGF1) (Harris and Chess 2003). Application of pegylation has also been extended for engineering long circulating particulate colloidal delivery systems such as liposomes and nanoparticles, leading to FDA approval of pegylated liposomal formulation of doxorubicin (Alza). Although pegylation of the particulate delivery systems has been proven to be a promising and effective technology to modify the pharmacokinetics and tissue distribution in a way to confer long circulation time in blood and enhanced accumulation in target tissues, there are still problems to be overcome. These include the eventual recognition and clearance of the pegylated particulate systems by the RES upon intravenous injection, and the accelerated blood clearance and altered biodistribution of the pegylated delivery systems after repeated administration (Moghimi and Hunter 2001). Therefore a further understanding of the immunological factors that control the pharmacokinetics and biological behavior of the pegylated particles is crucial for the design of a particulate delivery system with an optimal therapeutic performance.

### Liposomes Encapsulating Gene Therapeutics

A variety of approaches have been described for preparation of liposomes encapsulating gene therapeutics. As with other macromolecular therapeutics, it has been a challenge to encapsulate large DNA molecules in small liposomes. Although most of the procedures employed cationic lipid such as phosphatidylserine to facilitate encapsulation of negatively charged gene therapeutics, a majority of approaches still suffers from low encapsulation efficiency. The liposomal delivery systems are also subject to RES uptake and show in vivo behavior similar to other particulate systems. However, PEG-stabilized liposomes, which proved to be promising approaches to improve pharmacokinetics of small molecular weight drugs, have also been applied to prepare long circulating gene delivery systems suitable for in vivo application. The PEG-stabilized liposomes encapsulating plasmid DNA (Wheeler, Palmer et al. 1999; Shi and Pardridge 2000) or antisense ODN (Stuart, Kao et al. 2000) have been successfully applied for in vivo DNA delivery.

Approaches of condensing the DNA using polycationic polymer followed by encapsulation into liposomes have also been reported. LPDII was prepared by first condensing plasmid DNA with polylysine and then entrapping the complexes into folate-targeted anionic liposomes for tumor-specific gene transfer (Lee and Huang 1996). Plasmid DNA also was condensed with PEI and entrapped into endothelial targeted liposomes, resulting in so called ‘artificial virus-like particles’ (Muller, Nahde et al. 2001). Although the liposomes encapsulating polycation/DNA complexes showed promising in vitro gene transfer efficiency, no in vivo data have been reported.

### Polyethylenimine (PEI) as a Non-Viral Gene Delivery Vector

Among polycationic polymers, the polyethyleneimines (PEI) have been widely explored for the gene delivery due to their high gene transfer efficiency. The high gene transfer efficiency of PEIs mainly depends on their characteristic chemical structure. PEIs contain one amino group per every two carbons (ethylene group) and a significant fraction of the amino groups is protonated at physiological pH, resulting in high positive charge density. Due to the high positive charge density, PEIs form dense nano-sized particulate complexes with negative charged DNA by electrostatic interactions. The PEI/DNA complexes take overall positive charge and interact with negatively charged components of cell membranes and enter cells by endocytosis. The positively charged PEI/DNA complexes can enter the cells by nonspecific adsorption-mediated endocytosis while the condensed DNA in the complexes is protected from enzymatic degradation. Upon endocytosis, the PEIs are subject to further protonation as the endosomal compartment becomes acidic. Further protonation of PEI by capturing protons, the so called ‘proton sponge’ mechanism (Boussif, Lezoualc'h et al. 1995; Akinc, Thomas et al. 2005), leads to osmotic swelling and subsequent endosome disruption. Hence, gene delivery using PEI is based on (i) condensation of the negatively charged DNA into compact particles by electrostatic interactions, thus protecting the DNA from enzymatic degradation, (ii) endocytosis of the particles into the cells and (iii) release of the DNA from endosomes via the ‘proton sponge’ mechanism. Due to these favorable properties it achieves high transfection efficiency. Consequently, PEI and PEI-derivatives have been widely explored in gene delivery research as non-viral vectors for plasmid DNA or oligonucleotides (Boussif, Lezoualc'h et al. 1995; Kircheis, Wightman et al. 2001; Vinogradov, Batrakova et al. 2004; Akinc, Thomas et al. 2005).

### SUMMARY

The present invention relates to method for complexing a nucleic acid with a polymer, such as a cationic polymer, then encapsulating the complex in a liposome. This encapsulation method can serve to deliver the polymer/nucleic acid complexes systemically within an organism. The method may include a membrane extrusion step which allows for the formation of liposomes of a specific size. The liposomes may also be targeted to specific tissue types through the use of targeting molecules integrated into the lipsome, such as antibodies or ligands which recognize specific receptors. Polymer/nucleic acid complexes encapsulated in liposomes according to the disclosed method have shown significantly decreased clearance and prolonged circulation time as compared to the naked PEI/DNA complex after intravenous administration, and may also be appropriate for delivery of nucleic acids across the blood brain barrier.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic presentation of a possible embodiment of the invention;

FIG. 2 shows a representative fluorescence emission spectra of single labeled complexes in a preferred embodiment of the invention;

FIG. 3 shows representative fluorescence emission spectra of double labeled complexes in a preferred embodiment of the invention;

FIG. 4 shows quenching of fluorescence emission in PEI/dsODN complexes in a preferred embodiment of the invention;

FIG. 5 shows size measurement (A) and zeta potentials (B) of PEI/dsODN complexes in a preferred embodiment of the invention;

FIG. 6 shows fluorescence anisotropy (r) of the complexes between PEI and dsODN in a preferred embodiment of the invention;

FIG. 7 shows colloidal stability of PEI/dsODN/Anionic liposomes mixture by DLS size measurement in a preferred embodiment of the invention;

FIG. 8 shows distribution of PEI/dsODN/Anionic liposomes complexes on sucrose density gradient in a preferred embodiment of the invention;

FIG. 9 shows extraction of the micelle-like hydrophobic particles containing PEI/dsODN inside in a preferred embodiment of the invention;

FIG. 10 shows size distribution of intermediates (A) and final products (B) during encapsulation in a preferred embodiment of the invention;

FIG. 11 shows colloidal stability of the lipid-coated PEI/dsODN complexes in PBS in a preferred embodiment of the invention;

FIG. 12 shows fluorescence quenching of FL-dsODN in lipid-coated particles by KI in a preferred embodiment of the invention;

FIG. 13 shows SEC of lipid-coated PEI/dsODN complexes by reverse evaporation methods in a preferred embodiment of the invention;

FIG. 14 shows encapsulation efficiency of dsODN into bioPSL determined by SEC in a preferred embodiment of the invention;

FIG. 15 shows colloidal stability of bioPSL determined by DLS size measurement in a preferred embodiment of the invention;

FIG. 16 shows stability of the bioPSL particles in the presence of serum in a preferred embodiment of the invention;

FIG. 17 shows binding of streptavidin (SA) to the bioPSL particles (A) and stability of the binding (B) in a preferred embodiment of the invention;

FIG. 18 shows binding of 8D3-streptavidin conjugate (8D3SA) to the bioPSL particles in a preferred embodiment of the invention;

FIG. 19 shows in vitro cellular uptake of the bioPSL particles in brain endothelial cell (bEnd5) in a preferred embodiment of the invention;

FIG. 20 shows inhibition of VCAM-1 expression in bEnd5 by bioPSL particles in a preferred embodiment of the invention;

FIG. 21 shows the effect of PEG content on in vivo behavior of bioPSL particles in a preferred embodiment of the invention;

FIG. 22 shows concentration-time profiles of bioPSL, PEI2.7/dsODN and free dsODN in a preferred embodiment of the invention;

FIG. 23 shows organ distribution of dsODN after i.v. administration of bioPSL, PEI2.7/dsODN in a preferred embodiment of the invention;

FIG. 24 shows concentration-time profiles of antibody targeted 8D3bioPSL and non-targeted bioPSL in a preferred embodiment of the invention;

FIG. 25 shows organ distribution of dsODN after i.v. administration of bioPSL and 8D3bioPSL in a preferred embodiment of the invention; and

FIG. 26 shows stability of dsODN after i.v. bolus of 8D3bioPSL particles in a preferred embodiment of the invention.

### DETAILED DESCRIPTION OF PREFERRED EMBODIMENTS

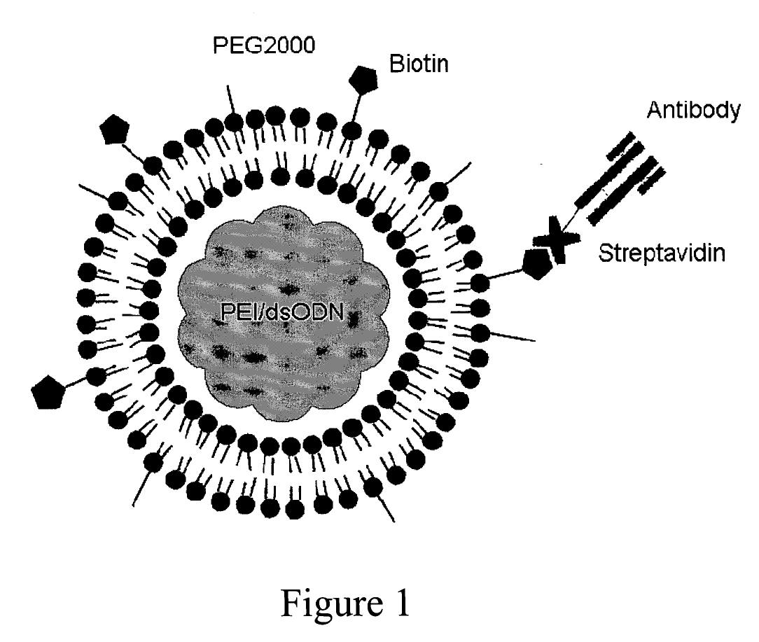

The current invention relates to liposome-encapsulated complexes comprising nucleic acids and polymers (“liposome-encapsulated nucleic acid/polymer complexes”) and methods for producing such complexes. A preferred embodiment of the invention includes a method for producing a complex comprising a nucleic acid and a polymer (“nucleic acid/polymer complex”), preferably including a negatively charged nucleic acid, most preferably DNA or RNA, and a positively charged polymer, most preferably PEI. This nucleic acid/polymer complex may then be encapsulated in a liposome, preferably a PEG-stabilized (polyethylene glycol-stabilized) liposome to form a liposome-encapsulated nucleic acid/polymer complex. One possible embodiment of the invention is shown schematically in FIG. 1.

Such liposome-encapsulated nucleic acid/polymer complexes have application as a drug delivery vehicle or as a means for delivering a nucleic acid to cells or to various sites in an organism, including across the blood brain barrier.

### Method for Producing Liposome-Encapsulated Nucleic Acid/Polymer Complexes Using Pre-Formed Anionic Liposomes

In a preferred embodiment of the invention, nucleic acid/polymer complexes are prepared from polymer, preferably 25 kDa polyethylenimine (PEI), and nucleic acid, preferably 20-mer double stranded oligodeoxynucleotides (dsODN), by fast addition of PEI solution to oligodeoxynucleotide (ODN) solution at amine/phosphate (N/P) ratio of about 6. The resulting mixture is incubated for about 10 min at room temperature.

In this embodiment of the invention, liposome-encapsulated nucleic acid/polymer complexes are prepared using pre-formed anionic liposomes. Multilamellar anionic liposomes, preferably comprising 1-Palmitoyl-2-Oleoyl-sn-3-[Phospho-rac-(1-glycerol)] (POPG), 1,2-Dilauroyl-sn-Glycero-3-Phosphoethanolamine (DLPE), 1,2-Dioleoyl-sn-glycero-3-phosphocholine (DOPC), and cholesterol, are prepared using the Low Temperature Trapping methods described in Huang, Buboltz et al. 1999, the entirety of which is hereby incorporated by reference. These multilamellar anionic liposomes would most preferably have a composition of approximately POPG/DLPE/DOPC/Cholesterol (2:3:3:2, w/w [where “w/w” refers to the dry weight of one lipid to the dry weight of the compared lipid]). The anionic liposomes are then extruded through a membrane with pore size of approximately 50 nm to obtain unilamellar liposomes with an approximate average diameter of 60 nm. The unilamellar liposomes are then mixed with the nucleic acid/polymer complexes described above to the yield a final N/P/POPG ratio of approximately (6:1:0.4) to (6:1:0.8).

### Method for Producing Liposome-Encapsulated Nucleic Acid/Polymer Complexes by Reverse Evaporation

Another embodiment of the invention includes the preparation of a nucleic acid/polymer complexes as described above, followed by encapsulation in liposomes using the reverse evaporation method. Similar methods have been described in Stuart and Allen 2000, the entirety of which is hereby incorporated by reference. Nucleic acid/polymer complexes are prepared as described above, and the resulting nucleic acid/polymer complexes with an N/P ratio of approximately 6 are used for encapsulation. Lipid, preferably anionic POPG (approximately 3.0 μmol) is diluted in approximately 1.0 ml CHCL₃, and approximately 2.08 ml MeOH is added, followed by approximately 1.0 ml of the preformed nucleic acid/polymer complexes (with approximately 100 μg corresponding to nucleic acid). After 30 minutes at room temperature, approximately 1.0 ml of CHCl₃ and approximately 1.0 ml of ddH₂O are added and then the tubes are centrifuged for approximately 7 minutes at about 830 g.

After removal of the aqueous phase, lipids, preferably 1-Palmitoyl-2-Oleoyl-sn-Glycero-3-Phosphocholine (POPC, approximately 6.7 μmol), Dimethyldioctadecylammonium Bromide (DDAB, approximately 0.2 μmol), 1,2-Distearoyl-sn-Glycero-3-Phosphoethanolamine-N-[Amino(Polyethylene Glycol)2000] (DSPE-PEG2000, approximately 0.3 μmol), and 1,2-Distearoyl-sn-Glycero-3-Phosphoethanolamine-N-[Biotinyl(Polyethylene Glycol)2000] (DSPE-PEG2000-Biotin, approximately 30 nmol) are added to the organic phase. Approximately 1 ml of approximately 10 mM HEPES (approximately 5% glucose, pH 7.4) was added, and the tube is vortexed vigorously and sonicated for 1 minute. CHCl₃ is then evaporated under vacuum on a rotary evaporator. The residual dispersion is extruded about 11 times through a membrane, preferably two stacks of 100 nm polycarbonate membrane, by using a hand held extruder.

### Method for Producing Liposome-Encapsulated Nucleic Acid/Polymer Complexes by Rehydration

In another embodiment of the invention, nucleic acid/polymer complexes are encapsulated in liposomes using the rehydration method. In this embodiment, lipids, preferably POPC (3.7 μmol), POPG (3.0 μmol), cholesterol (3.0 μmol), DSPE-PEG2000 (0.3 μmol) and DSPE-PEG2000-Biotin (0.03 μmol), are dissolved in chloroform. The chloroform is then removed by vacuum evaporation using a rotary evaporator (approximately 500 mmHg for about 4 hr). Nucleic acid/polymer complexes are prepared by separately diluting about 100 μl nucleic acid and about 90 μl polymer in 10 mM buffer, preferably containing 10 mM HEPES, 150 mM NaCl, 5% D-glucose, pH 7.4 (HBG), to a final volume of approximately 500 μl. The polymer solution is then added to the nucleic acid solution resulting in about 1 ml nucleic acid/polymer complexes (N/P approximately equal to 6) in buffer, preferably HBG. Approximately 1 ml of nucleic acid/polymer complexes is then added to the dried lipids and incubated at room temperature for a period of about 4 hr with intermittent mixing, resulting in a final lipid concentration of approximately 10 mM. The suspension is extruded multiple times, preferably 11 times, through a membrane, preferably a stack of two polycarbonate membranes of 100 nm pore size, employing a hand-held extruder. The resulting suspension is loaded onto a column, preferably a 1.0×30 cm Sepharose CL4B column, and then eluted with buffer, preferably 10 mM HEPES, 150 mM NaCl, pH 7.4 (HBS), at a concentration of approximately 10 mM at a flow rate of approximately 0.4 mL/min. The column eluents are monitored by on-line absorbance measurement at approximately 254 nm while 1 ml fractions are collected. The fractions containing liposome-encapsulated nucleic acid/polymer complexes are eluted at void volume.

### Liposome Encapsulated PEI/dsODN as a Vehicle

Further embodiments of the invention may comprise the use of the liposome-encapsulated nucleic acid/polymer complexes prepared using one of the methods described above for delivery of nucleic acid material or other therapeutic material within a cell or organism.

Another embodiment of the invention could comprise the incorporation of a targeting molecule into the liposome of a liposome-encapsulated nucleic acid/polymer complex as described above in order to direct the transport of the liposome-encapsulated nucleic acid/polymer complexes to a specific location in the cell or organism. The targeting molecule could comprise a ligand or an antibody, or any other molecule capable of directing the liposome-encapsulated nucleic acid/polymer complexes to a preferred location. The targeting molecule could also comprise a lipid conjugated to biotin, which could be used directly for targeting, or as a linker for attaching a further targeting molecule to the liposome-encapsulated nucleic acid/polymer complexes.

In another embodiment of the invention, the liposome-encapsulated nucleic acid/polymer complexes could be used to deliver nucleic acids or other materials to a cell or organism. This use could comprise delivery of a drug or therapeutic agent. This use may also include delivery of material across the blood-brain barrier. The described nanoparticulate system can be used for the in vivo delivery of DNA or RNA based drugs to cells in the body. Applications comprise the delivery of DNA- or RNA-based therapeutic agents, or oligonucleotides including antisense oligos, ribozymes, siRNA, transcription factor decoys, as well as gene therapy.

In a further embodiment of the invention, the size of the liposome-encapsulated nucleic acid/polymer complexes could be determined using a filtering device. This embodiment could further include the liposome-encapsulated complexes wherein the liposome-encapsulated nucleic acid/polymer complexes are smaller than approximately 130 nm in diameter.

### Abbreviations

ATP ADENOSINE TRIPHOSPHATE
AUC AREA UNDER THE CURVE
BBB BLOOD-BRAIN BARRIER
BPP BIOTINYLATED PEG-PEI
BP BASE PAIRS
BSA BOVINE SERUM ALBUMIN
CI CURIE
CNS CENTRAL NERVOUS SYSTEM
CPM COUNTS PER MINUTE
CRYO-TEM CRYO-TRANSMISSION ELECTRON MICROSCOPY
° DEGREE CELCIUS
DA DALTON
DLPE DILAUROYLPHOSPHATIDYLETHANOLAMINE
DLS DYNAMIC LIGHT SCATTERING
DMEM DULBECCO'S MINIMAL ESSENTIAL MEDIUM
DOPC 1,2-DIOLEOYL-SN-GLYCERO-3-PHOSPHOCHOLINE
DPM DECAYS PER MINUTE
DMSO DIMETHYLSULFOXIDE
DNA DEOXYRIBONUCLEIC ACID
DS DOUBLE STRANDED
DSODN DOUBLE STRANDED OLIGODEOXYNUCLEOTIDE
EDTA ETHYLENE DIAMINE TETRA ACETATE
EEA1 EARLY ENDOSOME ANTIGEN 1
FPLC FAST PROTEIN LIQUID CHROMATOGRAPHY
FRET FLUORESCENCE RESONANCE ENERGY TRANSFER
G-CSF GRANULOCYTE COLONY-STIMULATING FACTOR
HR HOUR
HMW HIGH MOLECULAR WEIGHT
HPLC HIGH PRESSURE LIQUID CHROMATOGRAPHY
ICA INTERNAL CAROTID ARTERY
% ID PERCENTAGE OF INJECTED DOSE
IGG IMMUNOGLOBULIN G
I.V. INTRAVENOUS
KB KILO BASES
KDA KILO DALTON
KSV QUENCHING CONSTANTS
LMW LOW MOLECULAR WEIGHT
LPS LIPOPOLYSACCHARIDE
LSCM LASER SCANNING CONFOCAL MICROSCOPY
MAB MONOCLONAL ANTIBODY
MIN MINUTE
MPS MONONUCLEAR PHAGOCYTE SYSTEM
MW MOLECULAR WEIGHT
M MICRO
MRNA MESSENGER RNA
NM NANOMETER
NF-KB NUCLEAR FACTOR-KB
NHS N-HYDROXY-SUCCINIMIDE
N/P RATIO AMINE/PHOSPHATE RATIO
ODN OLIGODEOXYNUCLEOTIDES
PAGE Polyacrylamide Gel Electrophoresis
PCR POLYMERASE CHAIN REACTION
PE PHOSPHATIDYLETHANOLAMINE
PEG POLYETHYLENE GLYCOL
PEI POLYETHYLENIMINE
PFA PARAFROMALDEHYDE
POPG PALMITOYLOLEOYLPHOSPHATIDYLGLYCEROL
% Q PERCENT QUENCHING
RHB RINGER-HEPES BUFFER
RES RETICULOENDOTHELIAL SYSTEM
RNA RIBO NUCLEIC ACID
RPM RTATIONS PER MINUTE
RT ROOM TEMPERATURE
RT-PCR REAL TIME PCR
SA STREPTAVIDIN
SEC SIZE EXCLUSION CHROMATOGRAPHY
SIL STEALTH IMMUNOLIPSOMES
SS SINGLE STRANDED
SSL STERICALLY STABILIZED LIPOSOMES
TBE TRIS BORATE EDTA
TCA TRICHLORO ACITIC ACID
TE TRIS EDTA
TEM TRANSMISSION ELECTRON MICROSCOPY
TFR TRANSFERRIN RECEPTOR
TMR TETRAMETHYLCARBOXYLRHODAMINE
TNF TUMOR NECROSIS FACTOR
U UNITS
UV ULTRAVIOLET
V VOLT
VCAM-1 VASCULAR CELL ADHESION MOLECULE-1
V0 ESTIMATED INITIAL BLOOD VOLUME OF DISTRIBUTION

Nomenclature: The PEG-stabilized liposome encapsulating PEI/dsODN complexes was denoted by bioPSL, or bioPSL(PEI/dsODN) with the encapsulated molecules inside parenthesis when necessary.

### Example 1

### Fluorescence Resonance Energy Transfer (FRET)

Double-labeled complexes (TMR-PEI/FL-dsODN) were prepared with 5′-fluorescein labeled dsODN (FL-dsODN) and tetramethylrhodamine (TMR-PEI), and single labeled complexes (PEI/FL-dsODN) with FL-dsODN and unlabeled PEI. 20 μg of dsODN and the desired amounts of PEI were diluted separately in HEPES buffer (10 mM HEPES, 5% glucose, pH 7.4) to a final volume of 500 μl. After 10 min incubation at room temperature, the PEI solutions were then transferred to the dsODN solution by fast addition and vortexed immediately. After additional 10 min incubation at room temperature, 1 ml of HEPES buffer was added to a final volume of 2 ml. The amounts of PEI were calculated from the desired amine/phosphate (N/P) ratio assuming that 43.1 g/mol corresponds to each repeating unit of PEI containing one amine and 330 g/mol corresponds to each repeating unit of ODN containing one phosphate. The amounts of fluorescence dyes in all preparations were kept constant and the TMR to FL molar ratio in double-labeled complexes was 1.

Single labeled complexes (PEI/FL-dsODN) and double-labeled complexes (TMR-PEI/FL-dsODN) were prepared at varying N/P ratio while maintaining the amounts of dyes constant. The fluorescence intensities were measured using a spectrofluorometer at excitation wavelength 480 nm and emission scanning from 510 to 610 nm with a slit width of 10 nm. The decreases in FL emission intensity at 518 nm as a result of fluorescence quenching were expressed as % Quenching (% Q) according to

$$\% Q = 100 \times ( 1 - \frac{{I}_{( N / P = n )}}{{I}_{( N / P = 0 )}} )$$

, where I_{(N/P=0)} and I_{(N/P=n)} are the FL emission intensity of free FL-dsODN at N/P=0 and FL emission intensities of complexes at N/P=n. The % Q by energy transfer (% Q_{energy transfer}), also known as efficiency of fluorescence resonance energy transfer (E), was calculated by subtracting the % Q value of the single labeled complexes (% Q_{complex}) from the % Q value of the double labeled complexes (% Qₜₒₜₐₗ) with the corresponding N/P ratio:

% Q_{energy transfer}=% Qₜₒₜₐₗ−% Q_{complex}

From the calculated E, the average distance (R) between the two fluorophores in the double-labeled complexes was determined by the equation

$$E = \frac{{R}_{0}^{6}}{( {R}_{0}^{6} + {R}^{6} )}$$

, where R₀ is the Förster radius of the FL-TMR dye pair, i.e., the distance at which energy transfer for the donor-acceptor pair is 50% of maximum.

Fluorescence quenching by complex formation was monitored by preparing single labeled complex PEI/FL-dsODN and then measuring decreases in emission intensities of FL. FRET was monitored by preparing double labeled complex TMR-PEI/FL-dsODN and then measuring decreases in emission intensities of FL. Assuming that the decreased emission intensities of FL in double labeled complexes represents the sum of fluorescence quenching by complex formation and FRET, the FL emission intensity decrease in double labeled complexes was subtracted from the intensity decrease in single labeled complexes at corresponding N/P ratio, thus obtaining the contribution of FRET to the total quenching.

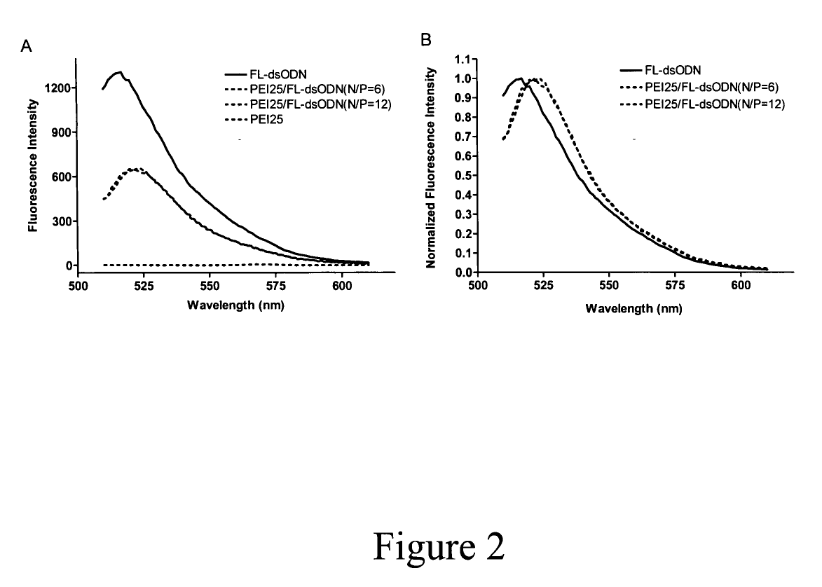

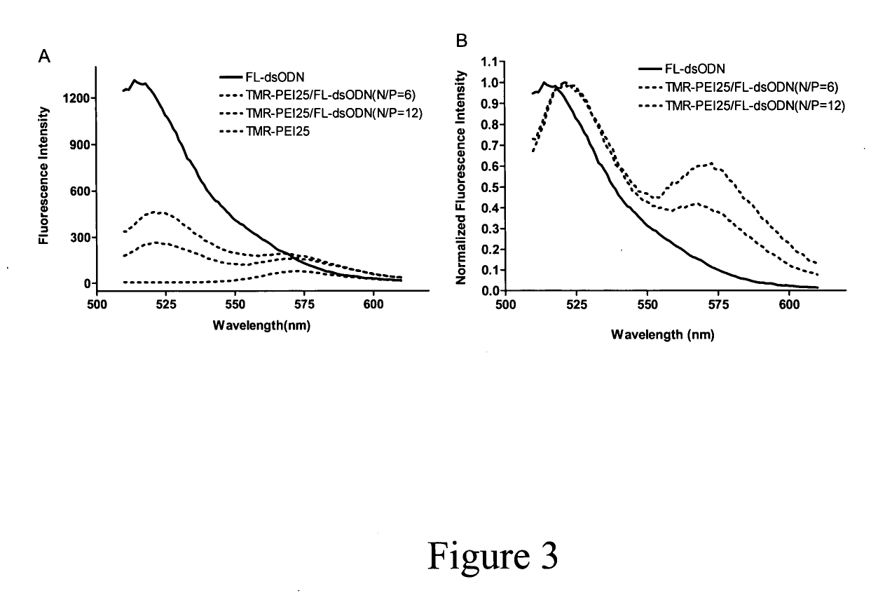

Emission spectra of the single labeled complex (FIG. 2) showed significant decreases in FL emission and red shift in the emission maximum of FL as compared to the spectra of free FL-dsODN. Emission spectra of the double-labeled complexes (FIG. 3) also showed the same degree of red shift in the emission maximum of FL, but even more decreases in the FL emission, and increases in the TMR emission as compared to single labeled complex at the same N/P ratio.

The quenching of FL emission in single labeled complexes is mainly a consequence of complex formation, causing some changes in spectral properties of FL as supported by the red shift of emission maximum. The additional quenching of FL emission in double labeled complexes is a consequence of energy transfer between FL and TMR in close proximity as supported by the sensitized emission of TMR.

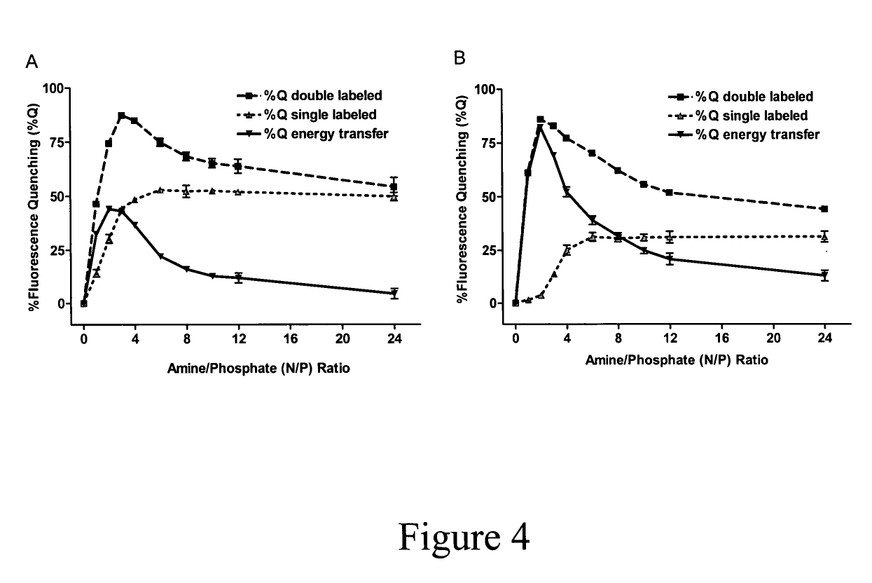

The fluorescence quenching technique has been applied to monitor complex formation between ODN and several polymers and it was observed that the emission intensity of rhodamine conjugated to ODN decreased and reached a plateau at the decreased level upon complex formation with polyethylene glycol (PEG)-modified PEI (Van Rompaey, Engelborghs et al. 2001). Consistent with these observations, the % Quenching of FL emission intensities in single labeled complexes PEI/FL-dsODN increased significantly up to an N/P ratio of 4 and reached a plateau above an N/P ratio of 6 (FIG. 4).

The fluorescence quenching in the single labeled complexes may be explained by self-quenching between FL in close proximity upon complex formation, but also indicates static quenching of FL by complex formation in ground state, thus suppressing excitation of FL and changing the spectral properties of FL, which becomes noticeable by the shift in the emission maximum of FL. The plateau at higher N/P ratio indicates that the structure of complexes does not change further with increasing N/P ratio as described previously (Van Rompaey, Engelborghs et al. 2001). The constant quenching also suggests that the amount of PEI in complexes reaches saturation, leaving a significant fraction of PEI free at higher N/P ratios. The quenching curve of PEI25/FL-dsODN showed a similar profile as that of PEI2.7/FL-dsODN as a function of N/P ratio, but reached plateau at a higher quenching level (≈55%) as compared to PEI2.7/FL-dsODN (≈30%). This indicates that the interaction of PEI25 with dsODN is different from that of PEI2.7, thus resulting in a different degree of condensation and different structure of complexes. In order to obtain distance data of PEI/dsODN complex, double labeled complexes (TMR-PEI/FL-dsODN) were prepared at various N/P ratio and FL emission intensity was measured in the same way as for single labeled complexes. The double-labeled complexes showed significantly higher quenching in FL emission than single labeled complexes at all N/P ratios, with maximum at N/P ratio 2. After the maximum, the quenching curves of the double-labeled complexes declined, apparently converging to the plateau of single labeled complexes. Assuming that total quenching in double labeled complex represents the sum of static quenching by complex formation and dynamic quenching by energy transfer, the higher quenching in double labeled complexes than single labeled complexes is due to introduction of acceptor dye (TMR) and thus energy transfer between dyes in close proximity upon complex formation.

The corrected quenching value (% Q_{energy transfer}=% Q_{double labeled}−% Q_{single labeled}) representing efficiency of energy transfer (% E) was used to estimate the Förster radius, the distance (R) between donor and acceptor in the double-labeled complexes TMR-PEI/FL-dsODN at which energy transfer is 50% of the maximum by the equation

$$E = \frac{{R}_{0}^{6}}{( {R}_{0}^{6} + {R}^{6} )}$$

The average distance between the donor and acceptor in double-labeled complexes TMR-PEI25/FL-dsODN at N/P ratio 3, where % E reached maximum, was estimated to be 5.72±0.03 nm (mean ±SE, n=3). This is significantly different from the average distance in TMR-PEI2.7/FL-dsODN of 4.25±0.01 nm (mean ±SE, n=3; unpaired t-test: p<0.0001), indicating that the difference in molecular weight and chemical structure of PEI leads to different spatial proximity and thus different conformation upon complex formation with dsODN.

FRET could be measured by monitoring either the decrease in the donor emission intensity (donor quenching) or the increase in the acceptor emission intensity (acceptor sensitization). Determination of FRET by the acceptor sensitization often requires complex formulations and rather high amounts of acceptor, with a donor to acceptor ratio of 1:5 (Itaka, Harada et al. 2002), necessitating high dye substitution and thus introducing the risk of altering the physical properties of the molecules of interest. In this study, one dsODN molecule contained one FL molecule and TMR was conjugated to about 2% of amino groups in PEI, corresponding to approximately 10 TMR per PEI25 and one TMR per PEI2.7. Donor to acceptor (FL:TMR) molar ratio was adjusted to 1:1 and decreases in donor (FL) emission were monitored instead of enhanced acceptor (TMR) emission. The complexes were excited at 480 nm rather than the absorbance maximum of FL at 488 nm to avoid direct excitation of TMR and minimize its background fluorescence.

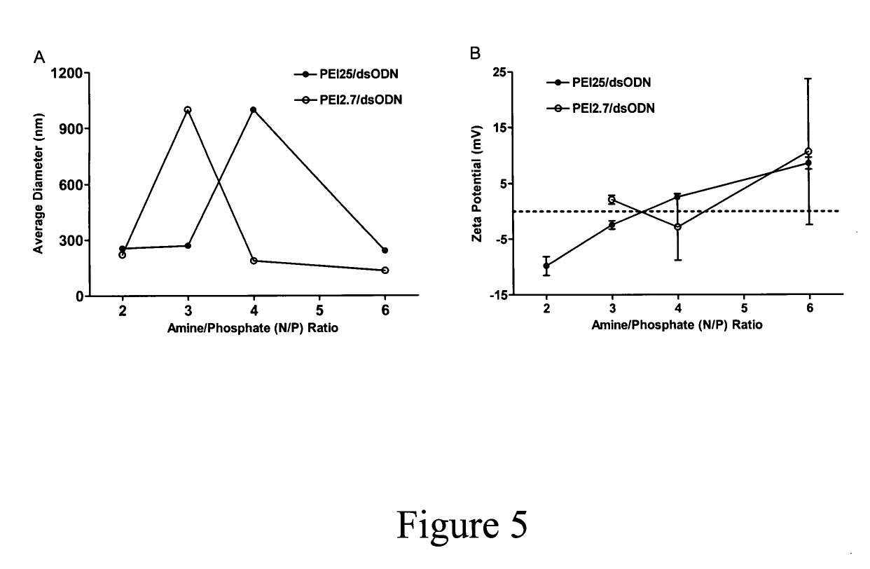

The decline of quenching after the maximum is likely due to multiple sources. First, since the total amounts of dyes were kept constant, at high N/P ratio, an increasing amount of dyes is not in close proximity and thus an increasing amount of TMR-PEI does not participate in complex formation, thus lessening the contribution of energy transfer to total quenching. The diminishing contribution of energy transfer is also supported by the convergence of the quenching curves for single labeled and double labeled complex as the N/P ratio increases. Second, as the N/P ratio gradually increases, the net charge of the complexes goes through transitions from strongly negative at low N/P ratio to neutral, then to strongly positive at high N/P ratio. A necessary condition for FRET is that the dipole moments of donor and acceptor need to align during the lifetime of the donor's excited state. Strong local electric fields, either at high N/P ratio (by PEI) or at low N/P ratio (by dsODN), can restrict the rotation of dipole moments, which can change the orientation factor and result in low FRET efficiency. However, between these two extremes, at N/P ratio, at which the electric field inside the complex is neutralized, the dipole moments have the highest rotational freedom and FRET reaches the highest efficiency. This mechanism would also contribute to a FRET maximum. It has been shown that in lipid bilayers with cholesterol, the packing of molecules (even without strong electric field) can sharply change the orientation factor and FRET efficiency (Parker, Miles et al. 2004). This explanation of an electrostatic effect is supported by our Zeta potential and size measurements of the complexes as a function of the N/P ratio. The Zeta potential approaches zero around the N/P ratio of 3 or 4. The measurement uncertainty is likely due to the dispersion of particle size in the samples. In addition, the average size of the complexes clearly peaks around N/P ratio of 3 in the PEI2.7/dsODN system, and around a N/P ration of 4 in the PEI25/dsODN system. This indicates that weak electrostatic repulsion promotes the aggregation of the complexes (FIG. 5).

In this study, the Förster radius, R₀ was not determined from experimental data but assumed to be 5.5 nm, a value used by several other groups working with DNA complexes (Edelman, Cheong et al. 2003; Wang, Gaigalas et al. 2003). It should, therefore, be noted that the distances calculated here might not represent absolute values. Nevertheless, the estimates provide useful information for comparison of the complexes generated with PEI of different molecular weight. The significant difference in Förster radius between PEI25/dsODN and PEI2.7/dsODN indicates that the difference in molecular weight and chemical structure of PEI leads to different spatial proximity and thus different conformation upon complex formation with dsODN. A likely explanation for the discrepancy lies in the known difference in branching between PEI25 and PEI2.7, as reflected in the ratios of primary:secondary:tertiary amines. PEI25 has a ratio of 1:1:1, indicating a higher degree of branching compared to a ratio of 1:2:1 for PEI2.7 (von Harpe, Petersen et al. 2000). The more branched structure and higher molecular weight of PEI25 imposes a higher degree of conformational constraint on the amino groups within an individual PEI molecule. As a consequence, not all of the acceptor dye substituents in TMR-PEI25, which contained approximately 10 dye molecules per PEI molecule, may be able to optimally approach the donor dye molecules (FL) in the dsODN at minimum distance. Such conformational restriction would be less significant in TMR-PEI2.7, which contained one dye molecule per PEI molecule, making each acceptor dye spatially independent.

### Example 2

### Steady-State Fluorescence Anisotropy Study

Two different series of single labeled complexes were prepared with FL-dsODN and unlabelled PEI (PEI/FL-dsODN), or unlabeled dsODN and TMR-PEI (TMR-PEI/dsODN) as described above, while maintaining the amounts of fluorescence dyes constant. PEI/FL-dsODN complexes were prepared with constant amounts of dsODN (20 μg) and varying amounts of PEI. TMR-PEI/dsODN complexes were prepared with constant amounts of PEI (18 μg) and varying amounts of dsODN.

Single labeled complexes, PEI/FL-dsODN and TMR-PEI/dsODN, were prepared at varying N/P ratios while maintaining the amounts of dyes constant. Fluorescence intensities were measured using a T-mode C61/2000 spectrofluorometer. The excitation wavelength was set to 480 nm, and emission intensities were scanned from 500 to 600 nm for PEI/FL-dsODN complexes. The excitation wavelength was set to 550 nm, and emission intensities were scanned from 570 to 630 nm for TMR-PEI/dsODN. The steady-state anisotropy (r) was then calculated as

$$r = \frac{( {I}_{vv} - g \times {I}_{vh} )}{( {I}_{vv} + 2 g \times {I}_{vh} )}$$

, where Iᵥᵥ is emission intensity of vertically polarized light and Iᵥₕ is emission intensity of horizontally polarized light, when excitation light is vertically polarized (Shinitzky and Barenholz 1978). The parameter “g” (g-factor) relates the relative sensitivity of the two emission channels and can be obtained as g=Iₕᵥ/Iₕₕ, with the polarization of excitation set to horizontal (Parker, Miles et al. 2004).

Complexes between PEI and dsODN were studied by steady state fluorescence polarization anisotropy with single labeled complexes of either PEI/FL-dsODN or TMR-PEI/dsODN. When fluorescent-labeled small molecules are excited with polarized light, the emitted light is depolarized due to fast rotational movement of the molecules. However, when the small molecules participate in complex formation, the rotational movement slows down resulting in less depolarization of the emitted light and increased anisotropy (Kakehi, Oda et al. 2001). Since the complex formation between PEI and dsODN leads to a significant change in rotational mobility of these molecules, the change in rotational mobility can be monitored by fluorescence anisotropy and used to characterize the complexes.

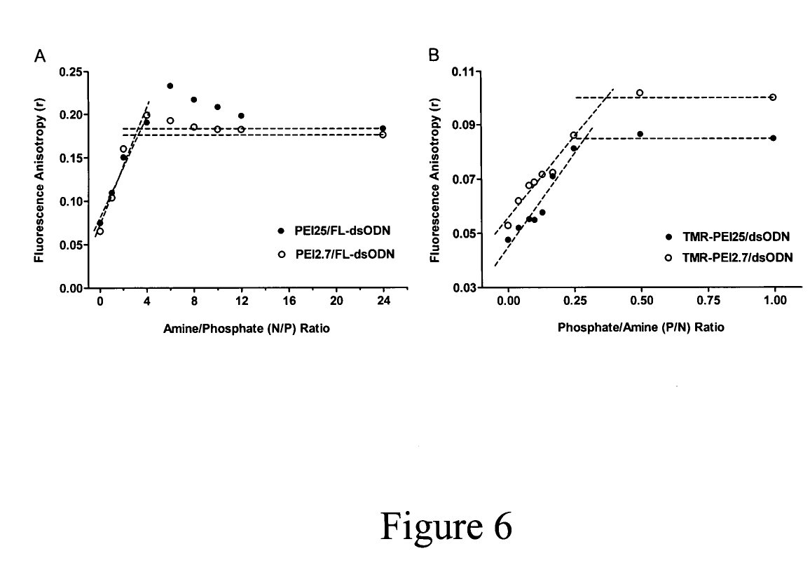

In the present study, two single labeled complexes, PEI/FL-dsODN with varying amount of PEI and TMR-PEI/dsODN with varying amount of dsODN, were prepared while maintaining the amount of dyes constant in each preparation. Steady-state anisotropy (r) of the complexes was determined and then plotted as a function of N/P ratio (PEI/FL-dsODN) or P/N ratio (TMR-PEI/dsODN). The anisotropy of the TMR-PEI/dsODN complexes showed linear increase over the lower P/N ratios and then reached a plateau. The anisotropy leveled out at a P/N ratio of about 0.25 (TMR-PEI25) and 0.3 (TMR-PEI2.7), demonstrating saturation. The saturation at higher P/N ratio indicates that PEI2.7 has more amino groups available for interaction with ODN phosphate groups than PEI25, which is consistent with the lower average distance in PEI2.7/dsODN complexes observed in the FRET experiments. Anisotropy of PEI/FL-dsODN complexes showed a similar profile as TMR-PEI/dsODN complexes, with an initial increase over N/P ratios 0 to 4 (FIG. 6).

Assuming a linear proportion between anisotropy and bound fraction of TMR-PEI up to the saturation point, linear regression analysis of these data points resulted in highly significant correlation coefficients (r²=0.9576 and 0.9314 for PEI2.7 and PEI25, respectively). In the present study, the fractions of bound PEI molecules in each preparation were calculated, as 43% (for PEI2.7) and 62% (for PEI25) at P/N ratio of 0.17 (corresponding to N/P ratio 6).

In this study, the fractions of bound PEI molecules in each preparation were calculated, as 43% (for PEI2.7) and 62% (for PEI25) at P/N ratio of 0.17 (corresponding to N/P ratio 6). Based on fluorescence correlation spectroscopy measurements, Clamme et al. reported that only 14% of PEI is bound in complexes prepared with PEI25 and plasmid DNA at N/P ratios of either 6 or 10, and that the average complex contains 30 PEI and 3.5 plasmid DNA molecules (Clamme, Azoulay et al. 2003). The discrepancy in bound fraction (62% vs. 14% at N/P ratio 6 for PEI25) at the same N/P ratio is probably due to the size difference of the DNA molecules (20 bp dsODN vs. 5.8 kbp plasmid DNA). The larger size of the plasmid DNA may cause conformational restriction for phosphate groups in the DNA molecules and thus limit interaction with amino groups in PEI molecules, leading to partial charge neutralization (N/P=0.4 based on complex composition of 30 PEI and 3.5 DNA molecules, or N/P=0.8 based on 14% binding). In comparison, small size of dsODN (20 bp) would cause relatively less constraint and thus make the phosphate groups more accessible to amino groups for charge interactions, leading to complete charge neutralization (N/P=3.7 based on 62% binding). It is, however, unexpected that the bound fraction as measured with correlation spectroscopy did not show a significant change when the N/P ratio increased from 6 to 10 (Clamme, Azoulay et al. 2003), which is in contrast to the present data where the bound fraction decreased as N/P ratio increased (P/N ratio decreased).

The Zeta potential changed from negative to positive as N/P ratio increased, approaching zero around the N/P ratio of 3 or 4. The average size of the complexes showed peaks around N/P ratio of 3 in the PEI2.7/dsODN system, and around a N/P ration of 4 in the PEI25/dsODN system, indicating that weak electrostatic repulsion promotes the aggregation of the complexes.

### Example 3

### Encapsulation of PEI/dsODN Complexes by Pre-Formed Anionic Liposomes

PEI/dsODN complexes were prepared from 25 kDa PEI and 20-mer double strand ODN (dsODN) by fast addition of PEI solution to ODN solution at N/P ratio 6. After 10 min incubation at RT, the size distribution of the complexes was measured using dynamic light scattering (DLS). Multilamellar anionic liposomes with the composition of POPG/DLPE/DOPC/Cholesterol (2:3:3:2, w/w) were prepared at final total lipid concentration of 10 mM using the Low Temperature Trapping methods (Huang, Buboltz et al. 1999) and then extruded through membrane with pore size of 50 nm to obtain unilamellar liposomes, resulting in unilamellar liposomes with an average diameter of 60 nm. The unilamellar liposomes were then mixed with the preformed PEI/dsODN complexes to the final N/P/POPG ratio of (6:1:0.4) and (6:1:0.8). The mixtures were analyzed with respect to size distributions and stability in saline solution by DLS and sucrose density gradient ultracentrifugation.

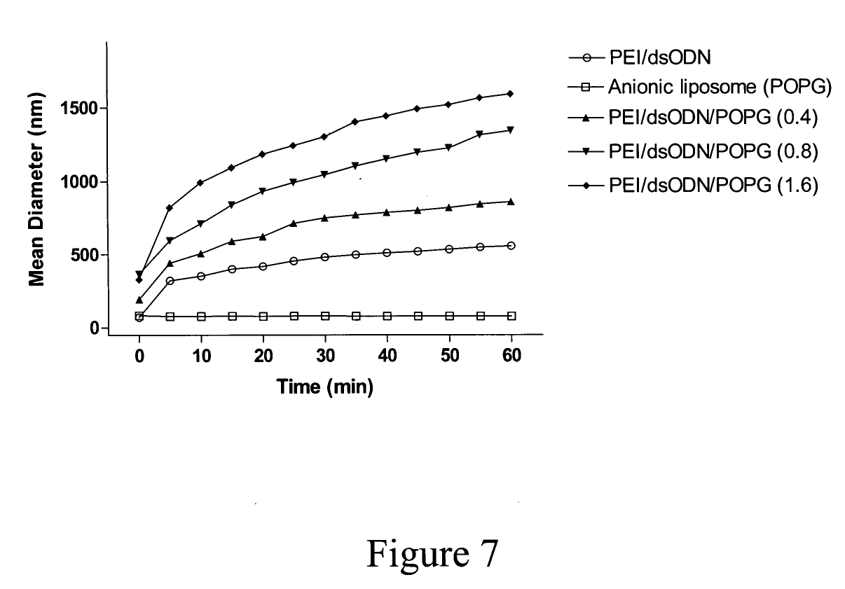

As an approach to encapsulate PEI/dsODN complexes within liposomes, PEI/dsODN complexes were prepared from 25 kDa PEI and 20-mer double stranded oligodeoxynucleotides (dsODN) at NP ratio 6 and then mixed with unilamellar liposomes containing 20% (w/w) anionic lipid POPG. After 30 min incubation at RT, size distribution of the mixtures was analyzed with DLS. The colloidal stability of the mixtures in saline solution was also determined by size measurement. Addition of anionic liposomes to PEI/dsODN complexes caused association of PEI/dsODN with anionic liposomes as shown by increases in mean diameters with a heterogeneous bimodal distribution, with mean diameters of 80 nm and 300 nm. The mixtures in saline showed continuous increase in mean diameter, indicating aggregation and thus incomplete encapsulation within lipid layers (FIG. 7).

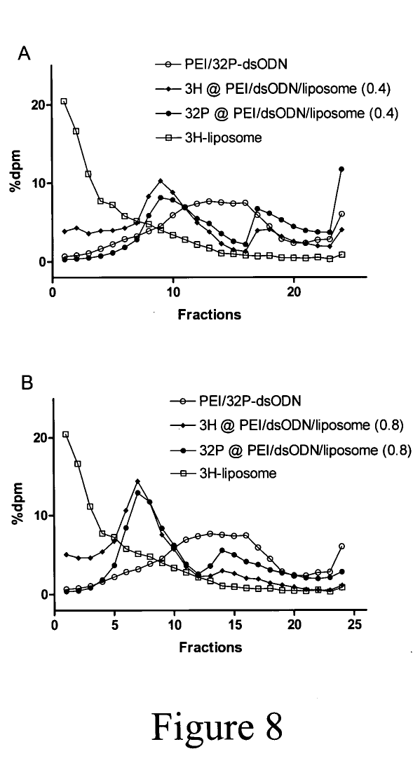

The association between PEI/dsODN complexes and anionic liposomes was also shown by accumulation of lipid and dsODN in the same fractions, between the fractions of free liposomes and PEI/dsODN complexes alone, on sucrose density gradient centrifugation (FIG. 8).

### Example 4

### Encapsulation of PEI/dsODN Complexes by Reverse Evaporation

The procedure for active entrapping of antisense ODN into pegylated liposomes, described by Allen's group (Stuart and Allen 2000), was modified and applied to entrap preformed PEI/dsODN complexes into liposomes. Anionic lipid POPG was used to extract positively charged PEI/dsODN complexes. For the preparation of PEI/dsODN complex, 90 μg of PEI and 100 μg of dsODN were separately diluted into 500 μl of 10 mM HBG (5% glucose, pH 7.4). After 10 minutes at room temperature, the PEI solution was transferred to the dsODN solution by fast addition and vortexed briefly. After 10 more minutes at room temperature, the resulting PEI/dsODN complexes with N/P ratio 6 were used for encapsulation. Anionic POPG (3.0 μmol) was diluted in 1.0 ml CHCl₃ and 2.08 ml MeOH was added followed by 1.0 ml of the preformed PEI/dsODN complex (100 μg corresponding to dsODN). After 30 minutes at room temperature, 1.0 ml of CHCl₃ and 1.0 ml of ddH₂O were added and then the tubes were centrifuged for 7 minutes at 830 g.

After removal of the aqueous phase, POPC (6.7 μmol), DDAB (0.2 μmol), DSPE-PEG2000 (0.3 μmol), and DSPE-PEG2000-Biotin (30 nmol) were added to the organic phase. 1 ml of 10 mM HEPES buffer (5% glucose, pH 7.4) was added, and the tube was vortexed vigorously and sonicated for 1 minute. CHCl₃ was then evaporated under vacuum on a rotary evaporator. The residual dispersion was extruded 11 times through two stacks of 100 nm polycarbonate membrane by using a hand held extruder.

The complexes were also characterized with respect to colloidal stability in PBS, protection of the encapsulated dsODN from external environment by fluorescence quenching and DNAse I digestion. In addition, the complexes were visualized by transmission electron microscopy (TEM).

The procedure described for active entrapping of antisense ODN into pegylated liposomes (Stuart and Allen 2000), was modified and applied for entrapping preformed PEI/dsODN complexes into PEG-stabilized liposomes. In this study, the complex between PEI and 20-mer dsODN prepared in aqueous buffer was combined with anionic phospholipid POPG in organic monophase. Overall organic environment and electrostatic interaction between POPG and PEI/dsODN complex resulted in a hydrophobic inverted micellar structure with PEI/dsODN complex inside. The hydrophobic particles were recovered in the organic phase after phase separation. Reverse phase evaporation of the organic solvent after adding coating lipids POPC and DSPE-PEG2000 to form the outer leaflet around the hydrophobic particles resulted in stable aqueous dispersion, indicating formation of hydrophilic particles. The size measurement of the dispersion by dynamic light scattering showed an average diameter of 300 nm with wide distribution. The resulting dispersion was extruded 11 times through a stack of two 100 nm pore size polycarbonate membranes. The size of the dispersion was reduced to average diameter of 130 nm with narrow size distribution. The particles also showed colloidal stability and complete protection of dsODN from DNAse I digestion, suggesting stabilization of otherwise unstable PEI/dsODN complex and protection from the external phase by encapsulating the complex with PEG-stabilized liposomal structure. This structure was confirmed by electron microscopic analysis. TEM visualization of the particles with negative staining showed spherical structures with heavily stained PEI-dsODN core surrounded by a lightly stained lipid layer.

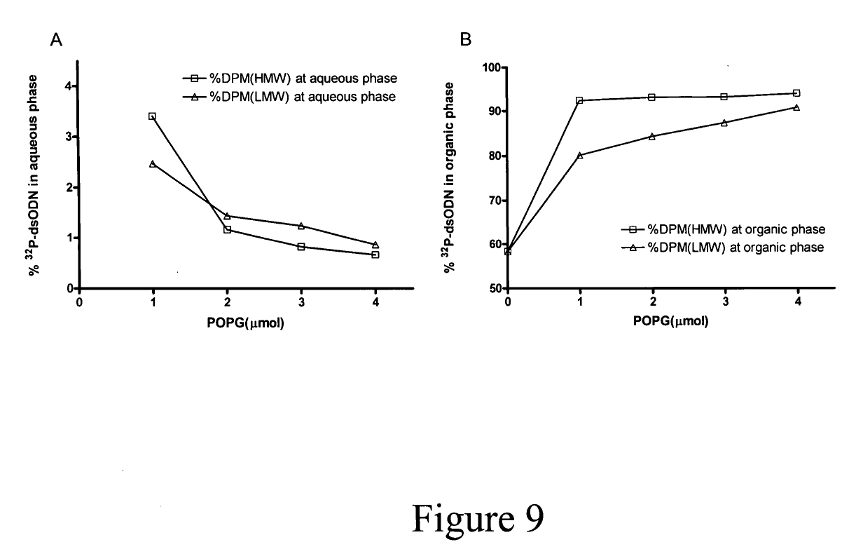

The PEI/dsODN complexes, which otherwise would be found in the aqueous phase, were almost completely recovered in the organic phase (FIG. 9), supporting formation of the hydrophobic particles. The resulting hydrophobic particles showed increased mean diameter as compared to PEI/dsODN complexes, indicating the formation of the hydrophobic particles.

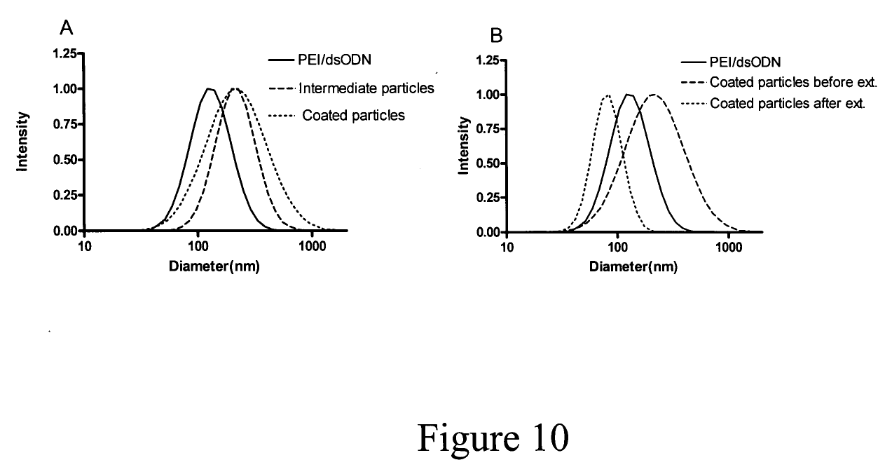

After addition of coating lipids followed by reverse evaporation of organic solvent, the coated particles showed increased mean diameter with wide distribution as compared to PEI/dsODN or the intermediate hydrophobic particles, indicating deposit of coating lipids around the hydrophobic particles. Membrane extrusion of the coated particles leads to narrow distribution and size reduction from 300 nm to 100 nm (FIG. 10).

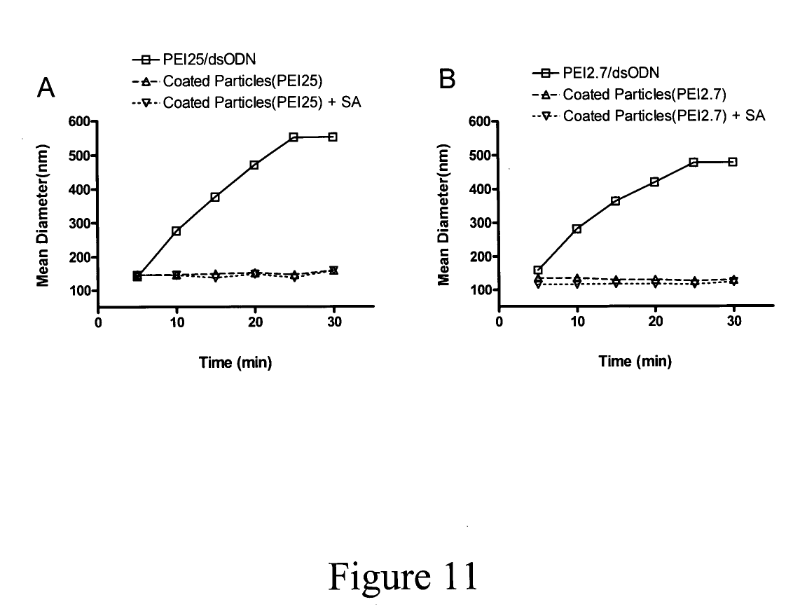

The colloidal stability of the coated particles was determined by size measurement. The mean diameters of the coated particles were measured at 5 min intervals, immediately after the particles were diluted into PBS. The mean diameter of the coated particles remained constant over 30 min while the naked PEI/dsODN complexes showed increasing mean diameter with time. The time-dependent increase of the mean diameter of the naked PEI-dsODN complexes indicates aggregation of the complexes due to surface charge screening effect, whereas the constant mean diameter of the coated particles indicates that the PEI/dsODN complexes are encapsulated inside the liposomes, leading to stabilization and protection of the otherwise unstable PEI/dsODN complexes from the external phase (FIG. 11). The colloidal stability of the particles was also measured in the presence of streptavidin (SA) at biotin:SA molar ratio 1. It is important to determine the stability of the coated particles in the presence of SA since the particles contain biotins at the distal end of PEG chains and SA has multiple binding sites for biotin, thus providing a possibility of cross-linking of the particles. The mean diameter of the coated particles remained constant in the presence of SA, indicating addition of SA to the particles does not cause cross-linking between the particles.

To demonstrate encapsulation of the PEI/dsODN complexes inside the lipid membrane, the lipid-coated particles were prepared with fluorescein-labeled dsODN. Concentrated KI solution was sequentially added to the coated particles and fluorescence emission intensities were measured. Since the encapsulated fluorescein-labeled dsODN would be protected from external quencher KI (Linnertz, Urbanova et al. 1997), the coated particles would show less quenching as compared to the naked PEI/dsODN complexes. The quenching constants (Ksv) were calculated using the equation

F₀/F=1+K_{SV}[Q]

where F₀=fluorescence emission intensity in the absence of KI, F=fluorescence emission intensity in the presence of KI, and [Q]=molar concentration of KI.

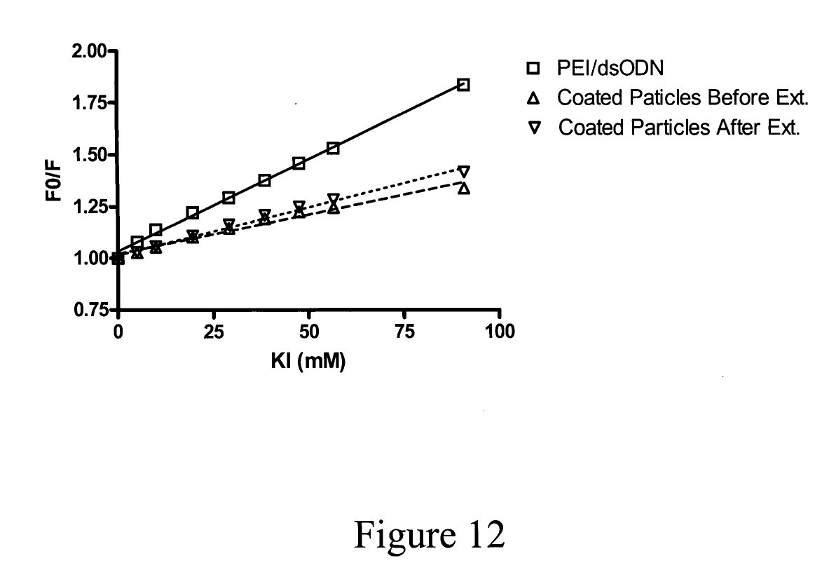

The lipid-coated particles showed a decreased quenching constant as compared to the naked PEI/dsODN complexes, indicating that the PEI/dsODN complexes are encapsulated inside the lipid membrane leading to protection from KI in the external phase (FIG. 12).

To demonstrate encapsulation of the PEI/dsODN complexes inside the lipid membrane, the coated complexes were also subjected to enzymatic degradation. The coated particles were incubated with DNAse I (100 U/mL) for 30 min at 37° C. The reaction was terminated by adding EDTA to a final concentration 5 mM. The resulting mixtures were treated with Triton X-100 at 1% final concentration and analyzed on a 1% agarose gel in TBE buffer. Free dsODN was completely degraded by the enzyme treatment. The naked PEI/dsODN complexes showed significant protection from enzymatic degradation, but failed to show complete protection. In contrast, the lipid-coated PEI/dsODN was completely protected from enzymatic degradation, supporting complete encapsulation of dsODN within the lipid membrane.

The coated particles were observed with conventional TEM to obtain morphological and structural information following application to silicon dioxide carbon-coated grids and negative staining with 1% uranyl acetate. The coated particles appeared as vesicles with lightly stained envelopes and heavily stained cores, probably representing lipid bilayers and PEI/dsODN complexes encapsulated within the lipid bilayers, respectively.

The particles before extrusion showed a broad distribution and irregularity in structure with 200˜300 nm diameter. The particles after extrusion showed a narrow distribution and uniform structure with ˜100 nm, which is consistent with size measurement by dynamic light scattering (DLS).

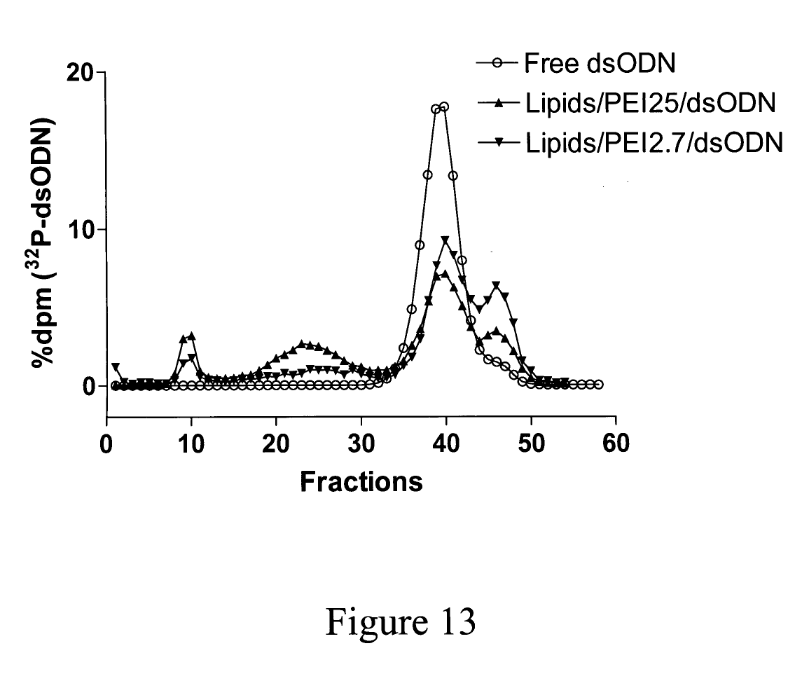

To determine the encapsulation efficiency of the procedure, the lipid-coated PEI/dsODN particles were prepared with ³²P-dsODN and subjected to SEC (Sepharose CL4B, 1×60 cm) with PBS as eluent (FIG. 13). Encapsulated ³²P dsODN was eluted at void volume with less than 10% for PEI25 and 5% for PEI2.7.

### Example 5

### PEG-Stabilized Liposomes Entrapping PEI/dsODN by Rehydration

POPC (3.7 μmol), POPG (3.0 μmol), cholesterol (3.0 μmol), DSPE-PEG2000 (0.3 μmol) and DSPE-PEG2000-Biotin (0.03 μmol) were dissolved in chloroform. The chloroform was removed by vacuum evaporation using a rotary evaporator (500 mmHg, 4 hr). PEI/dsODN complexes were prepared as described above. Briefly, 100 μl dsODN and 90 μl PEI were separately diluted in 10 mM HBG to a final volume of 500 μl, then the PEI solution was added to the dsODN solution resulting 1 ml PEI/dsODN complexes (N/P=6) in HBG. 1 ml of PEI/dsODN complexes was then added to the dried lipids and incubated at room temperature for 4 hr with intermittent mixing, resulting in a final lipid concentration of 10 mM. The suspension was extruded 11 times through a stack of two polycarbonate membranes of 100 nm pore size employing a hand-held extruder. The resulting suspension was loaded onto a 1.0×30 cm Sepharose CL4B column and then eluted with 10 mM HBS at a flow rate of 0.4 ml/min. The column eluents were monitored by on-line absorbance measurement at 254 nm while 1 ml fractions were collected. The fractions were also analyzed, when applicable, for other signals such as radioactivity or fluorescence. The fractions containing PEG-stabilized liposomes entrapping PEI/dsODN complexes were eluted at void volume and used for further studies. The PEG-stabilized liposome encapsulating PEI/dsODN complexes was denoted by bioPSL, or bioPSL(PEI/dsODN) with the encapsulated molecules inside parenthesis when necessary.

### Preparation and Physicochemical Characterization

20-mer dsODN containing NF-κB cis-element was condensed with PEI2.7 at N/P ratio 6. Lipid film containing the anionic lipid POPG was prepared with the lipid composition of POPC:POPG:Cho1:DSPE-PEG2000:DSPE-(PEG2000)Biotin (3.7:3.0:3.0:0.3:0.03, mol:mol). The anionic lipid film was then rehydrated in aqueous buffer containing positively charged PEI2.7/dsODN complexes. Assuming that about 25% of amino groups in PEI are protonated, the charge ratio of (+) in PEI2.7: (−) in dsODN: (−) in POPG is 1.5:1:3, i.e., negative charge in excess. The amount of anionic lipid POPG in lipid film was determined based on complete extraction of PEI/dsODN into organic phase by POPG as described in previously (section 3.3). The resulting suspension showed multimodal size distribution with a mean diameter≧300 nm. After extrusion through polycarbonate membrane with 100 nm pore size, the suspension achieved a narrow and unimodal size distribution with a mean diameter˜130 nm. Zeta potential measurement revealed that the positive charge of the naked PEI2.7/dsODN complexes (15.3±13.5) was completely shielded by anionic lipid membrane, resulting in slightly negatively charged particles (−4.06±0.71 mV), (Table 1).

TABLE 1
Size distribution and zeta potential of bioPSL particles

| | PEI/dsODN | bioPSL(before) | bioPSL(after) | 8D3SAbioPSL |
|---|---|---|---|---|
| Size distribution(nm) | 90.7 ± 50.6 | 351.0 ± 259.4 | 134.2 ± 32.57 | 142.7 ± 38.10 |
| Zeta potential(mV) | 15.3 ± 13.5 | NA | −4.06 ± 0.71 | −0.71 ± 0.94 |

Size distribution and zeta potential of bioPSL (before and after extrusion), and antibody conjugated bioPSL (8D3SAbioPSL) were determined in HBS by DLS. Data represent mean ±SD (n=3).

To obtain morphological and structural information, conventional transmission electron microscopy (TEM) analysis of the bioPSL particles was carried out and revealed vesicular structure with lightly stained envelopes and heavily stained cores, probably representing lipid membrane and PEI2.7/dsODN complexes, respectively. The particles before extrusion showed 200˜300 nm diameter with a broad distribution and irregularity in structure whereas the particles after extrusion showed ˜100 nm of diameter with a narrow distribution and uniform structure, which is consistent with size measurement by dynamic light scattering (DLS).

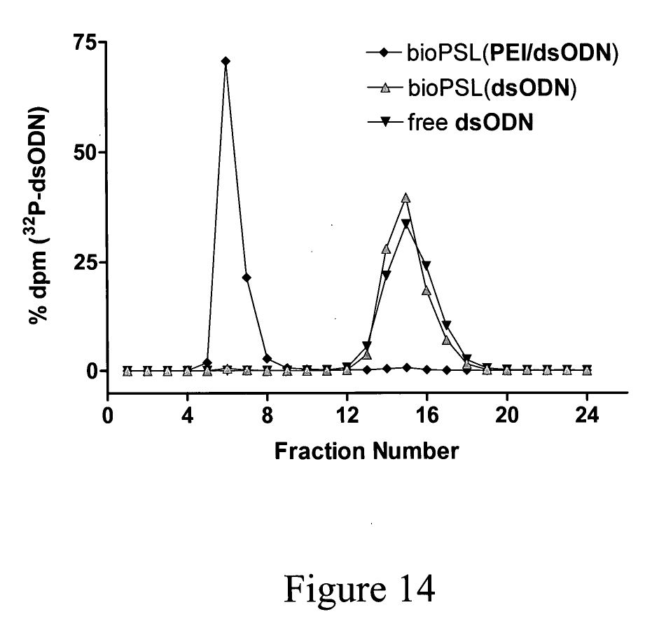

In order to determine the encapsulation efficiency of the procedure, the bioPSL particles were prepared with radioactively labeled dsODN (³²P-dsODN) and then subjected to SEC (Sepharose CL4B, 1×20 cm) with HBS as eluent. Recovery of ³²P-dsODN after membrane extrusion was ˜90%. After membrane extrusion, the resulting bioPSL particles were separated from free dsODN on a Sepharose CL4B column. More than 95% of ³²P-dsODN was eluted at void volume, representing encapsulated ³²P-dsODN (FIG. 14). The effect of precondensation by PEI on ³²P-dsODN encapsulation efficiency was demonstrated in comparison to a very low efficiency observed when free dsODN was subjected to the same encapsulation procedure without precondensation, supporting that precondensation of dsODN by PEI leads to a high encapsulation.

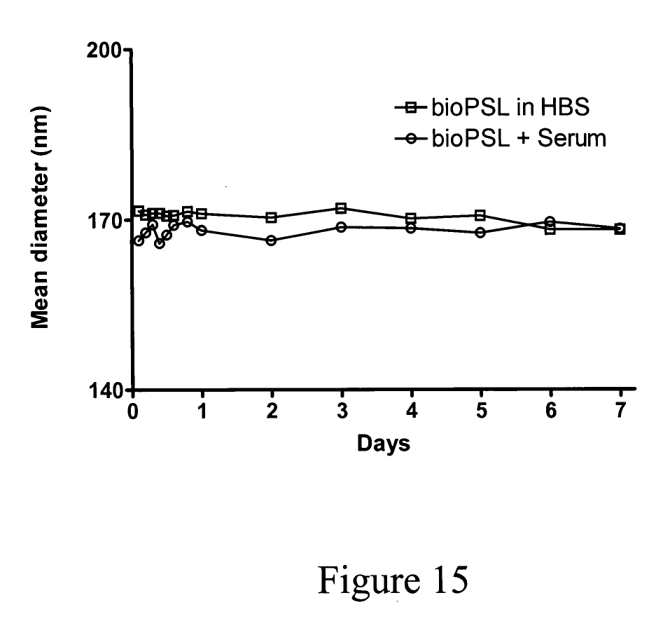

The liposomal delivery system should be sufficiently stable against the particle aggregation and loss of encapsulated therapeutic agents. The stability against aggregation can be determined by colloidal stability of the particles in physiological buffer. The colloidal stability of the bioPSL particles was determined by DLS size measurement. The mean diameter of the particles remained constant both in the absence and presence of serum for one week (FIG. 15). The colloidal stability also indicates that PEI/dsODN complexes, otherwise unstable and prone to aggregation, were encapsulated and thus stabilized by the PEG-stabilized lipid membrane.

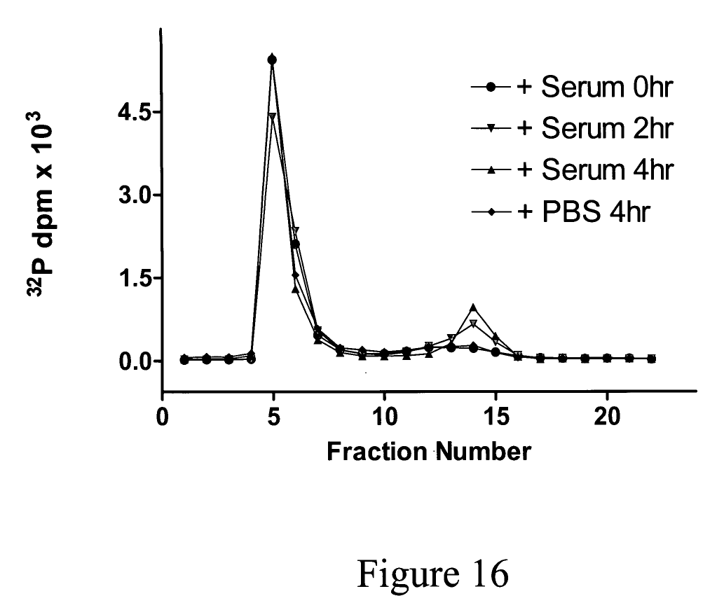

The stability against dissociation and loss of the entrapped dsODN was defined as the ability of PEG-stabilized liposome to retain the entrapped dsODN under physiological conditions. To demonstrate the stability of the bioPSL particles, the leakage of the entrapped ³²P-dsODN from the bioPSL(PEI/³²P-dsODN) particles in the presence of serum was determined by SEC. The bioPSL(PEI/³²P-dsODN) particles were incubated with mouse serum and then ³²P-dsODN leaked from the particles was separated by Sepharose CL4B. The amount of free ³²P-dsODN increased with incubation time (FIG. 16). After 4 hr incubation in the presence of serum, about 15% of dsODN was released from the bioPSL particles, whereas the leakage was insignificant after 4 hr incubation in the absence of serum, indicating some interaction of the bioPSL particles with serum.

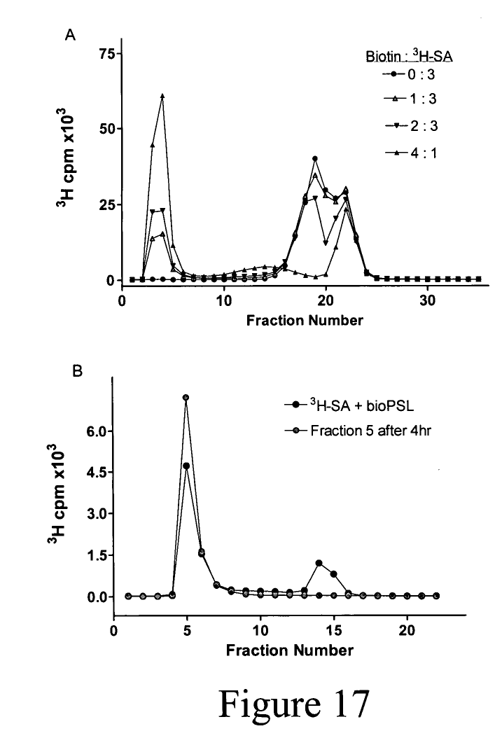

Binding of streptavidin (SA) to the bioPSL particles was also studied using SEC on a Sepharose CL4B column. After incubation of the bioPSL with ³H-SA at varying biotin:SA molar ratio, the bound ³H-SA was separated from free ³H-SA. The result indicates specific and concentration-dependent binding of ³H-SA to the bioPSL particles (FIG. 17). At biotin:SA molar ratio 4, SA was completely bound to the particles. The peak fraction (fraction 5) containing ³H-SA bound to bioPSL (³H-SA-bioPSL) from the first CL4B elution was again eluted through another CL4B column to determine the stability of the binding between ³H-SA and bioPSL particles. After 4 hr incubation of fraction 5 from the first CL4B separation, no free ³H-SA was found, suggesting that the binding between ³H-SA and bioPSL particles is stable.

The specific binding between the bioPSL particles and SA was also demonstrated by cryo-TEM analysis of the bioPSL particles after incubation with streptavidin-conjugated colloidal gold particles (Gold-SA). The bioPSL was incubated with Gold-SA at biotin:SA molar ratio 4 and then observed with cryoTEM. The bioPSL particles showed vesicular structure with diameter of ˜150 nm and uniform size distribution. The Gold-SA particles were found exclusively around the bioPSL while the presaturated Gold-SA showed random distribution, indicating specific binding of Gold-SA to the bioPSL particles. The specific binding of Gold-SA on the surface of the bioPSL particles confirmed that the biotins at the distal end of PEG chains on the surface of bioPSL are accessible to SA binding.

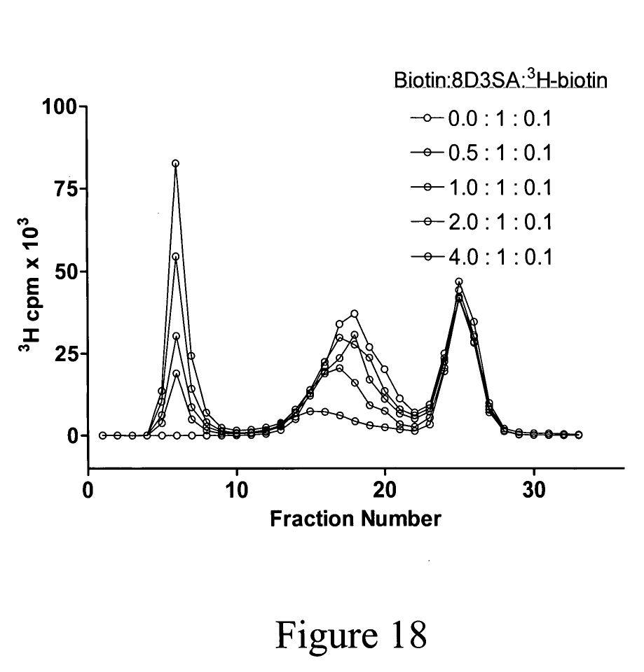

Binding of streptavidin-8D3 conjugate (8D3SA) to the bioPSL particles was also studied using Sepharose CL4B SEC. After incubation of the bioPSL with 8D3SA at varying biotin:SA molar ratio, ³H-biotin as a tracer was added to the mixture at SA: ³H-biotin molar ratio 10. The bound 8D3SA was separated from free 8D3SA with the result of specific and concentration-dependent binding of 8D3SA to the bioPSL particles (FIG. 18).

Most 8D3SA appears to bind to the biotin binding sites on bioPSL particles at biotin:streptavidin molar ratio 4:1. About 25% of 8D3SA binding to the particles at biotin:streptavidin molar ratio 1:1 suggests that only one biotin out of four in the particle is available to 8D3SA binding.

To demonstrate that the bioPSL particles contain both PEI and dsODN, the particles were also visualized and analyzed by LSCM. Double labeled bioPSL(TMR-PEI/A488-dsODN) particles were prepared and observed under LSCM. The bioPSL particles containing TMR-PEI and A488-dsODN were found as discrete particles with diameter of a few hundreds nanometer and the two dyes were perfectly colocalized. The detection and perfect colocalization of the two dyes confirms that the bioPSL particles contain both PEI and dsODN.

The double lableled bioPSL(TMR-PEI/A488-dsODN) particles were also investigated by FRET analysis to demonstrate that PEI and dsODN in the particles are in close proximity within nanometer range and thus forming compact complexes. The emission intensity of A488 in dsODN was increased after bleaching TMR in PEI. Increased donor (A488) intensity after acceptor (TMR) bleaching indicates energy transfer between the two dyes. The presence of FRET with ˜50% efficiency between the two dyes confirms that PEI and dsODN in the particles are in close proximity within a few nanometers.

### Example 6

### In Vitro Evaluation of bioPSL Particles in Brain Endothelial Cell (bEND5)

The bioPSL(PEI/³²P-dsODN) containing tracer ³²P-dsODN was prepared as described above and conjugated to 8D3SA at varying biotin:strepavidin molar ratio to a final concentration of 1 μM dsODN and used for transfection experiment. Mouse brain endothelial cell line bEnd5 was grown to confluency in 24 well plates at 37° and 5% CO₂. The cells were washed twice with 1 ml of PBS and preincubated with 500 μL of DMEM for 1 hr at 37°. Uptake of bioPSL(PEI/³²P-dsODN) was initiated by exchanging the medium with 500 μL of 8D3SA conjugated bioPSL(PEI/³²P-dsODN) in DMEM and incubating the cells for 0, 15, 30, and 60 min at 37°. Uptake was terminated by washing the cells twice with ice-cold PBS, followed by mild acid wash with 10 mM HEPES in DMEM (pH 3.0). The cells were then solubilized with 500 μL of 5% SDS in 1 M NaOH and assayed for radioactivity with liquid scintillation counting. The effect of serum on in vitro cellular uptake was also investigated. The 8D3 conjugated bioPSL(PEI/³²P-dsODN) at biotin:SA molar ratio 4 was incubated with the cells in DMEM containing 10% mouse serum for 60 min and then treated as above.

Cellular Uptake of bioPSL(PEI2.7/³²P-dsODN)

The bEnd5 cells were grown to confluency on cover slips. bioPSL(TMR-PEI/A488-dsODN) containing labeled dsODN and TMR labeled PEI were prepared and diluted to a final concentration of 1 μM dsODN into 0.5 ml of 10% FBS-supplemented DMEM cell culture medium and added to the cells. After 1 hr incubation at 37°, cells were washed four times with PBS. Cells were washed again with ice cold PBS containing 2% (w/v) paraformaldehyde. The coverslips with fixed cells were mounted with glycerol mounting media and examined on an inverted microscope (DMIR2) by laser scanning confocal microscopy.

For colocalization studies, single labeled bioPSL(PEI/TMR-dsODN) was prepared and added to the cells. The cells were treated as above and then blocked with 1% normal chicken serum for 30 min. After blocking, the cells were incubated with either goat anti-human polyclonal antibody (0.4 μg/mL) to early endosome antigen 1 or rabbit anti-human polyclonal antibody (0.4 μg/mL) to caveolin-1 in 10 mM PBS containing 0.05% sodium azide for 1 hr at RT. After washing with 10 mM PBS, the cells were incubated with Alexa Fluor-488 chicken anti-goat IgG (1 μg/mL) for EEA1 or Alexa Fluor 488-chicken anti-rabbit IgG for CAV1 (1 μg/mL) in 10 mM PBS containing 1% normal chicken serum and 0.05% sodium azide for 1 hr at RT. After 3 times washing with 10 mM PBS and counter-staining of nuclei with DRAQ5, the coverslips were mounted with glycerol mounting media and observed with LSCM. For control, the primary antibodies pre-incubated with 5 times blocking peptides were used and resulted in no staining.

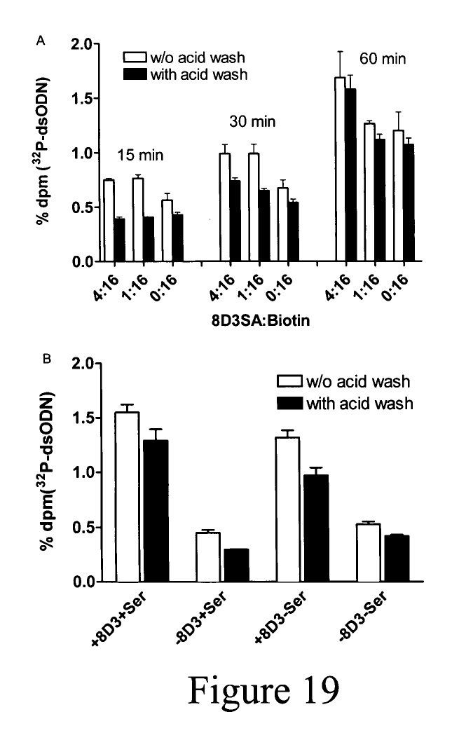

The in vitro cellular uptake of the bioPSL particles was studied in the mouse brain endothelial cell line bEnd5, which expresses the transferrin receptors (TfR). The bEnd5 cells were incubated with the bioPSL(PEI2.7/³²P-dsODN) particles targeted with TfR antibody 8D3 at varying ratio and then the amount of cell-associated ³²P-dsODN was measured (FIG. 19). The effect of serum on the cellular uptake was demonstrated by measuring the amount of cell-associated ³²P-dsODN after incubation of the cells in the presence of serum with bioPSL(PEI2.7/³²P-dsODN) conjugated to 8D3 at biotin:SA molar ratio 4 for 60 min.

The amount of cell-associated ³²P-dsODN increased as incubation time increased, in the absence or presence of the targeting antibody. However, conjugation of the targeting antibody 8D3 at the terminal ends of liposome-associated PEG chains in the bioPSL particles further increased the amount of cell-associated ³²P-dsODN. Since the cell-associated ³²P-dsODN comprises both internalized ³²P-dsODN and membrane-bound ³²P-dsODN, it is necessary to differentiate the membrane bound particles from internalized particles for quantitation of cellular uptake. The membrane-bound particles could be removed by mild acid wash, while the internalized particles remain cell-associated after the acid wash procedure. The amount of internalized ³²P-dsODN also increased with incubation time with further increase by conjugation of targeting antibody. The effect of serum on the cellular uptake of the bioPSL particles was insignificant as shown by no significant difference between the amount of cell-associated or internalized ³²P-dsODN in the absence and in the presence of serum.

### Example 7

### In Vitro Cellular Uptake of bioPSL Particles Analysed by LSCM

The cellular uptake of the bioPSL particles was visualized by LSCM. The fluorescent labeled bioPSL(TMR-PEI2.7/A488-dsODN) particles targeted with 8D3 were associated with and incorporated into the bEnd5 cells during the incubation period. The particles were found at the cell membranes and within the cells, primarily in the perinuclear region. Nuclear accumulation was not observed. Co-localization of TMR with A488 indicates that the particles were taken up in intact form and retained their integrity.

The internalization of the bioPSL particles was further investigated by colocalization of the particles with intracellular compartments such as early endosomes. Early endosomes are intracellular compartments that function in uptake and sorting of endocytosed proteins. Early endosome antigen 1 (EEA1) is a membrane protein known to colocalize with the TfR in early endosomes. The bEend5 cells were immunostained for EEA1 with polyclonal antibody after incubation with antibody-targeted single labeled bioPSL(PEI/TMR-dsODN) particles. EEA1 immunostaining of bEend5 cells showed cytoplasmic staining with vesicular compartments. The colocalization of the particles with EEA1 supports that the particles are internalized by TfR-mediated uptake.

However, the colocalization was not perfect as shown by no yellow color in the overlay images. The partial colocalization may be explained by the fact that the EEA1 is on the outside of the vesicles and the bioPSL particles are inside of the vesicles despite almost below optical resolution limit.

To obtain additional information on the internalization pathway of the bioPSL particles, the bEnd5 cells were immunostained for caveolae. Caveolae are 50-100 nm, nonclathrin-coated, flask-shaped plasma membrane microdomains that have been identified in most mammalian cell types, except lymphocytes and neurons and have been implicated in multiple functions including the compartmentalization of lipid and protein components that function in transmembrane signaling events, biosynthetic transport functions, endocytosis, potocytosis, and transcytosis. Caveolin, a 21-24 kDa integral membrane protein, is the principal structural component of caveolae (Cameron, Ruffin et al. 1997).

The BEnd5 cells were immunostained against CAV1 with a polyclonal antibody after incubation with antibody-targeted single labeled bioPSL(PEI/TMR-dsODN) particles. CAV1 immunostaining of bEnd5 cells shows membrane and cytoplasmic staining. Although the bioPSL particles showed intracellular localization, we could not find evidence of colocalization with CAV1 in our preparation.

### Example 8

### In Vitro Pharmacological Effect of bioPSL Particles

Mouse bEnd5 cells were grown to confluency in Dulbecco's modified Eagle's medium supplemented with 10% (v/v) fetal calf serum and incubated with bioPSL(PEI/dsODN) at a final concentration of 2 μM dsODN for 4 hr. At the end of incubation, cells were washed and fresh media were added. After additional 8 hr incubation, the mRNA expression of VCAM-1 was determined as described before (Fischer, Bhattacharya et al. 2005).

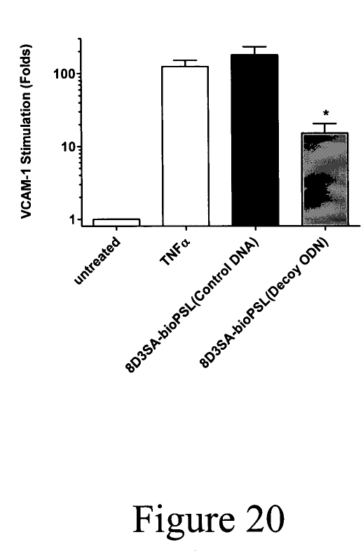

The pharmacological efficacy of the bioPSL particles with respect to inhibiting the NFκ-B pathway was tested in bEnd5 cells. Cells were treated with the antibody-targeted bioPSL particles after stimulation of NFκ-B pathway with TNF-α and the mRNA level of VCAM-1 was determined (FIG. 20).

VCAM-1 expression was hardly detectable in untreated cells, but remarkably increased by more than 100-fold in TNF-α stimulated cells. The VCAM-1 expression in TNFα stimulated cells was inhibited by 10-fold when treated with the targeted bioPSL(PEI2.7/dsODN) particles. In contrast, no inhibitory effect was observed when treated with the control formulation prepared with salmon sperm DNA (SSN). The sequence-specific effect on VCAM-1 expression confirms that the inhibition of VCAM-1 is mediated by the decoy dsODN.

### Example 9

### Pharmacokinetic Studies of the bioPSL(PEI2.7/³²P-dsODN)

Male Balb/c mice (20-30 g) were anesthetized by 1% isoflurane in 0.7 L/min N₂O, 0.3 L/min O₂ and catheterized with PE-10 in a retrograde direction into the right common carotid artery. bioPSL(PEI2.7/³²P-dsODN) was conjugated with 8D3SA at streptavidin:biotin molar ratio of 1:4 and diluted to a final concentration of 3.0 μM dsODN. 100 μl of the 8D3SA-bioPSL (PEI/³²P-dsODN) with ˜1 μCi ³²P activity (3.6 μg dsODN, 0.31 mg total lipids, 70 μg 8D3SA per animal) were injected into a jugular vein. Blood samples (30 μl) were taken through the catheter in the common carotid artery at 0.5, 1, 2, 5, 10, 20, 30, 60, 90, 120 min after intravenous bolus injection. The sample volume was replaced with PBS containing heparin (10 U/ml). After the last blood sampling the animals were sacrificed by decapitation and organ samples (brain, liver, kidneys, heart, lungs, spleen) were taken. The blood samples were centrifuged at 2000 g and 4° for 10 minutes to obtain plasma. The blood, plasma and organ samples were solubilized with Soluene-350 and diluted with Hionic-Fluor. Radioactivity of all samples was measured by liquid scintillation counting. The radioactivity was expressed as percentage of injected dose (% ID/g for organ, % ID/ml for blood and plasma). Organ distribution values were corrected for plasma volume of the corresponding organs using the equation,

$$\% ID \text{/} g = \frac{( {V}_{d} ( t ) - {V}_{0} ) \times {C ( t )}_{plasma}}{1000}$$

where V_{d}(t)=(dpm/g organ)/(dpm/μl plasma) at time t, V₀=plasma volume of the corresponding organs (μl/g), C(t)ₚₗₐₛₘₒ=plasma concentration (% ID/ml) at time t. The following V₀ values for the different organs were chosen: 9.3±1.1 μl/g (brain), 48.2±3.11l/g (heart), 170±16 μl/g (lung), 83±12 μl/g (kidney), 140±13 μl/g (liver), 114±12 μl/g (spleen) (Fischer, Osburg et al. 2004).

The pharmacokinetic parameters were determined by fitting concentration-time data to a biexponential disposition equation using non-linear regression,

C(t)=Ae^{-αt}+Be^{-βt}

where C(t) is the blood or plasma concentration, and α, β are the elimination rate constants. Secondary pharmacokinetic parameters (clearance, half lives, mean residence time, area under the curve) were calculated by standard formular.

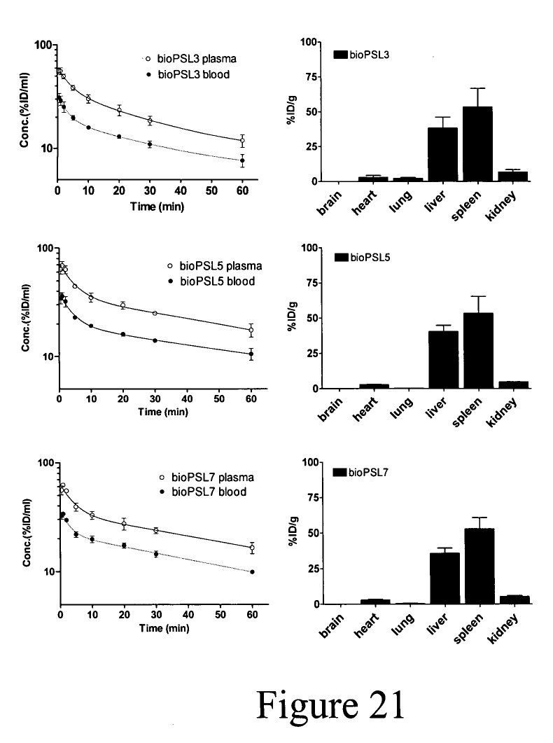

First, the effect of PEG content of bioPSL particles on in vivo behavior was investigated with bioPSL(PEI2.7/³²P-dsODN) particles with varying PEG content. bioPSL(PEI2.7/³²P-dsODN) containing 3%, 5%, 7% DSPE-PEG2000 of total lipids were prepared as above. The pharmacokinetic and biodistribution studies were performed with the bioPSL(PEI2.7/³²P-dsODN) particles in mice. The concentration time profiles of ³²P-dsODN radioactivity in whole blood and plasma following i.v. bolus administration of bioPSL(PEI2.7/³²P-dsODN) were obtained (FIG. 21). Area under the curve from 0 to 60 min (AUC_{—}60) values and other pharmacokinetic parameters were obtained by fitting the data to a biexponential disposition function. We did not test if triexponetial function would give a better fit. On the other hand, we do not have sufficient data points to try triexponential fitting. After 10 minutes, the ³²P-dsODN radioactivity was cleared from the plasma more slowly with half life of longer than 60 min.

Over the first 10 min after i.v. bolus, the radioactivity in the plasma decreased to about 50% of the initial concentration C(0) with half life of less than 5 min. The blood samples also showed similar concentration-time profile parallel to the plasma concentration-time curve. The estimated initial blood volume of distribution (V₀) values corresponds to the total blood volume in mice, indicating that the bioPSL particles apparently distribute initially in the blood space after i.v. bolus administration (Table 2). The plasma V₀ values was about half the corresponding value of the blood V₀, indicating the initial distribution in plasma with little binding to blood cells. The parallel concentration time curves for blood and plasma samples over the time period also indicates that the particles are retained in plasma compartment with no blood cell binding over the time period. No significant differences were observed among the particles with different PEG content.

TABLE 2
Pharmacokinetic parameters of bioPSL particles with varying PEG content

| | | A(% ID/ml) | t_{1/2, α}(min) | B(% ID/ml) | t_{1/2, β}(min) | V0(ml) | AUC_60 ((% ID/ml) × min) |
|---|---|---|---|---|---|---|---|
| bioPSL3 | Blood | 13.3 | 2.44 | 18.1 | 60.1 | 3.42 | 741 |
| | | (3.21) | (0.43) | (0.87) | (16.9) | (0.28) | (58.9) |
| | Plasma | 30.4 | 5.86 | 25.9 | 73.2 | 1.79 | 1440 |
| | | (1.50) | (0.46) | (2.56) | (6.82) | (0.11) | (135) |
| bioPSL5 | Blood | 19.9 | 3.38 | 18.5 | 91.7 | 2.67 | 944 |
| | | (3.61) | (0.50) | (1.72) | (23.7) | (0.33) | (14.0) |
| | Plasma | 37.2 | 3.96 | 34.9 | 46.2 | 1.43 | 1700 |
| | | (11.1) | (0.71) | (6.21) | (6.80) | (0.18) | (20.9) |
| bioPSL7 | Blood | 14.9 | 2.28 | 21.3 | 106 | 2.77 | 949 |
| | | (0.16) | (0.30) | (1.78) | (76.1) | (0.14) | (47.5) |
| | Plasma | 34.2 | 3.78 | 30.0 | 56.4 | 1.57 | 1570 |
| | | (4.56) | (1.20) | (5.62) | (4.74) | (0.10) | (104) |

Pharmacokinetic parameters (1 hr) were obtained by fitting concentration-time data to a biexponetial disposition equation. Data represent mean (SEM) (n ≧ 3).

Organ accumulation of ³²P-dsODN after i.v. bolus administration of the bioPSL particles is also shown. All data are expressed in units of percentage injected dose per gram (% ID/g). The radioactivity of ³²P-dsODN after administration of the bioPSL particles was found primarily in liver and spleen with very low uptake in other organs. The high level of accumulation in liver and spleen suggests that RES uptake still plays a major role in clearance of the particles from circulation. No significant differences in organ accumulation were observed among the particles with different PEG content and particles with 3% DSPE-PEG2000 were chosen for further studies.

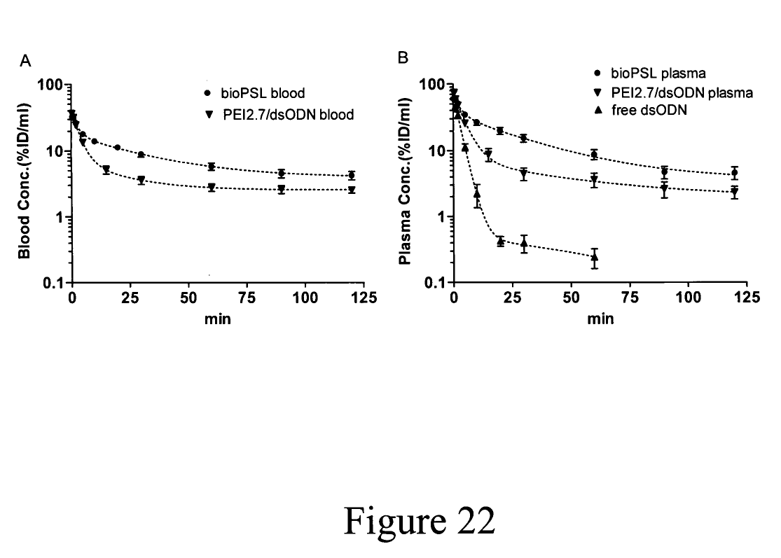

More pharmacokinetic and biodistribution studies were performed with bioPSL(PEI2.7/³²P-dsODN) prepared with 3% DSPE-PEG2000. The concentration-time profiles of ³²P-dsODN radioactivity in whole blood and plasma following i.v. bolus administration of the bioPSL(PEI2.7/³²P-dsODN) were obtained and compared to the concentration-time course of the free ³²P-dsODN and naked PEI/³²P-dsODN complexes (Fischer, Osburg et al. 2004). All pharmacokinetic parameters were obtained by fitting the data to a biexponential disposition function (Table 3). The bioPSL(PEI2.7/³²P-dsODN) demonstrated a biexponential plasma concentration-time curve (FIG. 22). Over the first 10 min after i.v. bolus, the radioactivity in plasma decreased to 60% of the initial concentration C(0) with a half life of 10.2±1.78 min. After 10 minutes, the ³²P-dsODN radioactivity was cleared from the plasma more slowly with a half life of 131.5±67.7 min. As compared to the naked PEI/dsODN complexes, the bioPSL particles showed significantly decreased plasma clearance. While less than 10% of injected dose remained in plasma after 10 min with the naked PEI/dsODN, more than 10% of the injected dose still remained in plasma after 60 min with the bioPSL particles. The bioPSL particles showed two time increased plasma AUC_{—}60 of 1221±122.0 whereas the free dsODN and naked PEI/dsODN complexes showed AUC_{—}60 of 213.1 and 589±77.3, respectively. The blood samples also showed similar concentration-time profile parallel to the plasma-concentration time curve. The estimated initial blood volume of distribution (V0) values of 2.85±0.23 corresponds to the total blood volume in the mouse, indicating that the bioPSL particles distribute initially in the blood space after i.v. bolus administration.

The plasma V0 of 1.76±0.08 was about half the corresponding value of the blood V0, indicating the initial distribution in plasma with little binding to blood cells. The parallel concentration-time curves for blood and plasma samples over 2 hr time period also indicates that the particles are retained in plasma compartment with no blood cell binding over the time period.

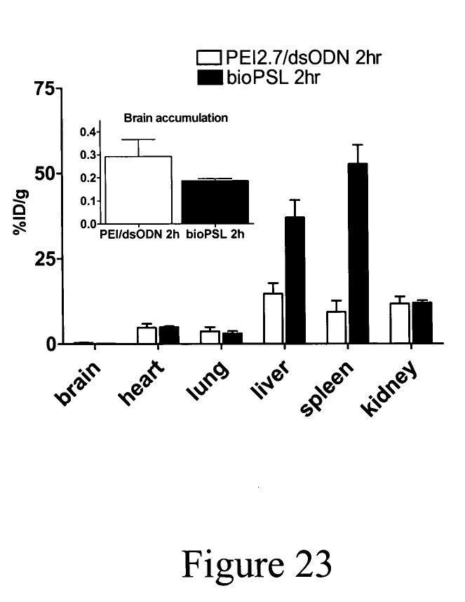

Encapsulation of dsODN within PEG-stabilized liposomes leads to a significant change in biodistribution of dsODN. Organ distribution of ³²P-dsODN 2 hr after i.v. bolus administration of the bioPSL particles is shown in comparison to naked PEI/dsODN (FIG. 23). The radioactivity of ³²P-dsODN dsODN after i.v. administration of the bioPSL particles was found primarily in liver, spleen and kidney with low level of accumulation in other organs. The bioPSL particles showed significantly higher accumulation in liver and spleen than the naked PEI/dsODN complexes. No significant differences were observed in other organs.

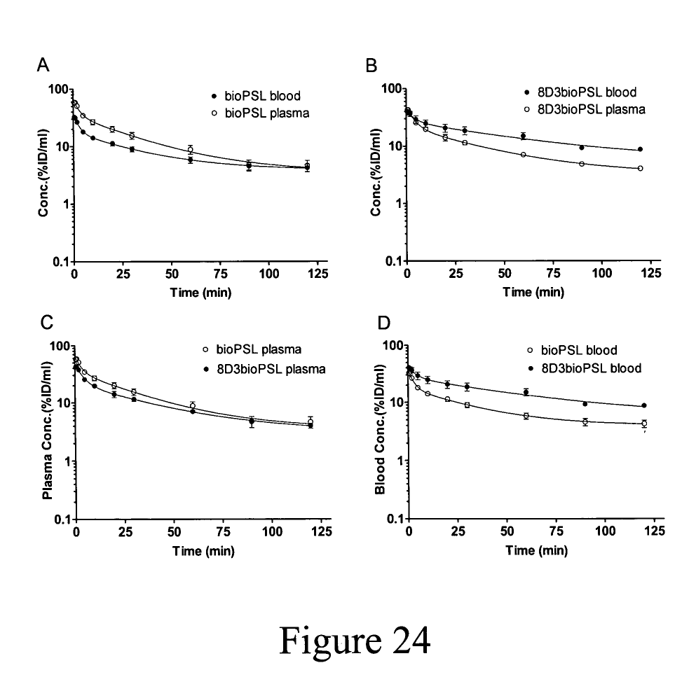

Equivalent pharmacokinetic studies were performed with antibody-targeted bioPSL(PEI2.7/³²P-dsODN) particles. The bioPSL(PEI2.7/³²P-dsODN) particles were conjugated to 8D3SA at biotin:SA molar ratio 4. The concentration-time profiles of ³²P-dsODN radioactivity in whole blood and plasma following i.v. bolus administration of the antibody targeted 8D3bioPSL were obtained and compared to the non-targeted bioPSL particles (Table 3). The antibody conjugation leads to significant changes in pharmacokinetic behavior of the bioPSL particles. The antibody targeted 8D3bioPSL particles also demonstrated biexponential plasma concentration-time curves after i.v. bolus. Over the first 5 min, the radioactivity in the plasma decreased to about 40% of initial concentration C(0) with half life of 3.73±0.57 min. After 5 min, the radioactivity was cleared from the plasma slowly with half life of 60.4±8.63 min. Less than 10% of the injected dose remained in plasma after 60 min. The targeted bioPSL particles were more rapidly cleared from plasma with AUC_{—}60 of 850±63.7 as compared to the non-targeted bioPSL particles with AUC_{—}60 of 1221±122.0. The antibody targeted 8D3bioPSL particles showed very slow blood clearance as compared to plasma clearance (Table 3 and FIG. 24). Over the first 5 min, the radioactivity in the blood decreased to about 50% of the C(0) with half life of 4.3±0.95 min. After 5 min, the radioactivity was cleared from blood very slowly with half life of 177±56.9 min. More than 20% of the injected dose still remained in the blood circulation after 60 min. The targeted bioPSL particles were cleared more slowly from blood with AUC_{—}60 of 1309±191.4 as compared to the non-targeted bioPSL particles with AUC_{—}60 of 655±49.0.

The plasma C(0) of the targeted particles was the same as the blood C(0) with plasma V0 of 2.14±0.10 and blood V0 of 2.38±0.24 whereas the plasma C(0) of the non-targeted particles was almost twice the blood C(0). The almost identical V(0) values of plasma and blood samples indicate that the targeted particles distribute in blood with significant binding to blood cells. The blood concentration-time curve diverging from the plasma concentration-time curve over 2 hr time period also indicates that the targeted particles remain bound to blood cells and release very slowly, resulting in higher blood AUC_{—}60 values of 1309±191.4 than plasma AUC_{—}60 values of 850±63.7.

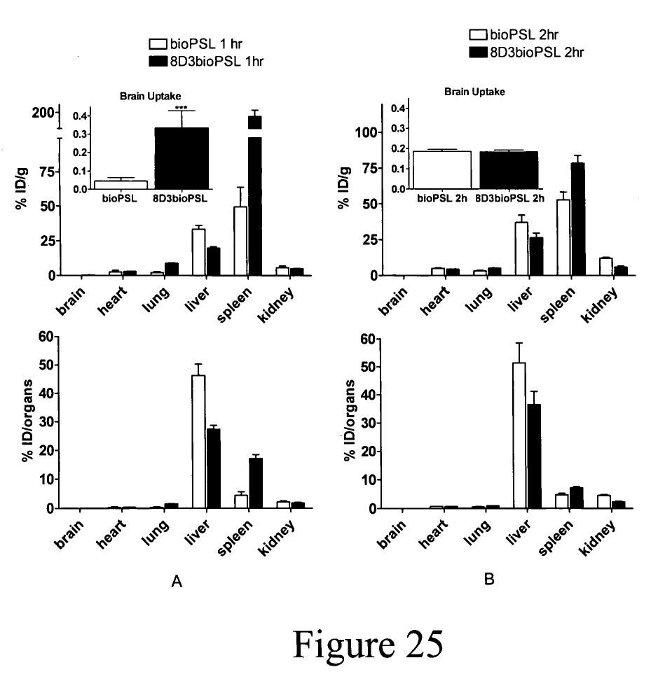

The antibody conjugation also caused significant changes in biodistribution of bioPSL particles. The organ distributions of ³²P-dsODN at 1 hr and 2 hr after i.v. bolus administration of non-targeted bioPSL and targeted 8D3bioPSL are compared (FIG. 25). For both bioPSL and 8D3bioPSL, the radioactivity of ³²P-dsODN at 1 hr after i.v. bolus administration was found primarily in liver and spleen with low levels in other organs, suggesting high RES uptake. However, the biodistribution was significantly modified by 8D3 conjugation.

TABLE 3
Pharmacokinetic parameters of free dsODN, PEI2.7/dsODN, bioPSL, and 8D3bioPSL

| | dsODN* | PEI/dsODN* | | bioPSL | | 8D3bioPSL | |
|---|---|---|---|---|---|---|---|
| | plasma | blood | plasma | blood | plasma | blood | plasma |
| A(% ID/ml) | 71.6 | 36.7 | 64.5 | 22.7 | 47.4 | 19.8 | 29.7 |
| | | (3.14) | (3.71) | (3.61) | (2.53) | (1.91) | (2.91) |
| B(% ID/ml) | 0.56 | 4.63 | 5.53 | 13.6 | 9.87 | 23.7 | 17.3 |
| | | (0.86) | (1.41) | (2.00) | (0.09) | (4.44) | (3.22) |
| t_{1/2, α}(min) | 1.84 | 2.7 | 3.4 | 2.7 | 10.2 | 4.38 | 3.73 |
| | | (0.48) | (0.46) | (0.41) | (1.78) | (0.95) | (0.57) |
| t_{1/2, β}(min) | 51.0 | 124 | 94.6 | 72.6 | 132 | 126 | 60.4 |
| | | (33.0) | (2.47) | (14.0) | (67.7) | (31.2) | (8.63) |
| V0(ml) | 1.38 | 2.46 | 1.43 | 2.85 | 1.76 | 2.38 | 2.14 |
| | | (0.13) | (0.09) | (0.23) | (0.08) | (0.24) | (0.10) |
| Cl(ml/min) | 0.43 | 0.13 | 0.10 | 0.08 | 0.02 | 0.02 | 0.06 |
| | | (0.03) | (0.02) | (0.01) | (0.01) | (0.01) | (0.01) |
| AUC_60 | 213.0 | 358 | 589 | 656 | 1221 | 1310 | 851 |
| (% ID/ml) × min | | (29.0) | (77.3) | (49.0) | (122) | (191) | (63.7) |
| AUC_120 | NA | 499 | 761 | 926 | 1750 | 2130 | 1158 |
| (% ID/ml) × min | | (54.2) | (117) | (67.9) | (184) | (296) | (56.2) |

Pharmacokinetic parameters (2 hr) were obtained by fitting concentration-time data to a biexponetial disposition equation. Data represent mean (SEM) (n ≧ 3).
*Adopted from Drug Metabolism and Disposition 2005, D Fisher et al.

The targeted 8D3bioPSL particles showed about 3 times increased accumulation in spleen and 50% decreased accumulation in liver as compared to the bioPSL. The 8D3SAbioPSL also showed significantly increased accumulation in lung. The 8D3bioPSL showed almost 10 times increased brain uptake as compared to the bioPSL, indicating that the increased brain uptake was mediated by targeting antibody 8D3. At 2 hr after i.v. bolus administration, the radioactivity of ³²P-dsODN was also found primarily in liver and spleen with low levels in other organs for both bioPSL and 8D3bioPSL. Although the targeted 8D3bioPSL particles still showed significantly increased spleen accumulation and decreased liver accumulation as compared to non-targeted bioPSL, the differences were attenuated as compared to 1 hr. The 8D3SAbioPSL also showed significantly decreased accumulation in kidney. Although brain accumulation for 8D3bioPSL was significantly different from bioPSL at 1 hr, the difference was abolished at 2 hr mainly due to increased accumulation of bioPSL.

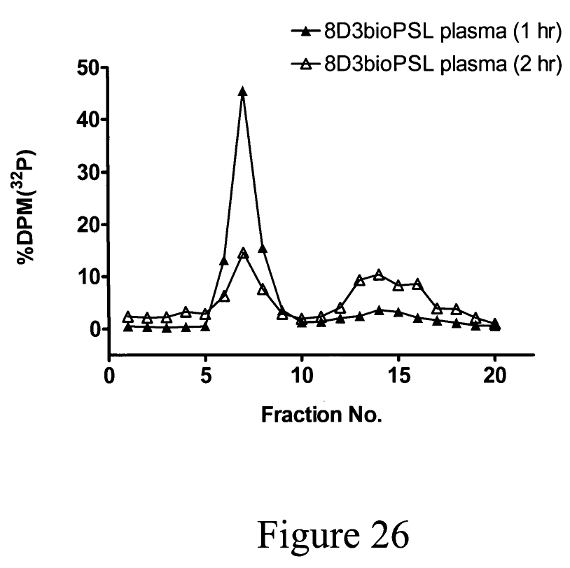

To determine the stability of dsODN in circulation, the plasma samples at 1 hr and 2 hr after i.v. administration of the targeted 8D3bioPSL(PEI/32P-dsODN) were loaded on Sepharose G50 column (PD-10) and eluted with HBS. The small nucleotides produced by degradation were separated from the intact dsODN. The intact dsODN was about 80% and less than 50% of total radioactivity at 1 hr and 2 hr, respectively (FIG. 26). This indicates that the dsODN was released from the particles and degraded by enzymatic digestion in blood circulation.

### Example 10

### Analysis of Intact dsODN from Brain and Blood after I.V. Bolus

Male Balb/c mice (20-30 g) were anesthetized by 1% isoflurane in 0.7 L/min N₂O, 0.3 L/min O₂. bioPSL(PEI2.7/³²P-dsODN) was conjugated with 8D3SA at streptavidin:biotin molar ratio of 1:4 and diluted to a final concentration of 3.0 μM dsODN. 100 μl of the 8D3SA-bioPSL(PEI2.7/³²P-dsODN) with ˜10 Ci ³²P activity (3.6 μg dsODN, 0.31 mg total lipids, 70 μg 8D3SA per animal) were injected into a jugular vein. At 10 min, 1 hr, and 2 hr after injection, blood samples (100 μl) were taken from jugular vein, and then the animals were perfused by transcardial perfusion of ice-cold saline TRIS buffer (TBS, 10 mM tris, pH 7.4). The cleared brains were removed and transferred to glass-Teflon tissue grinders. The dsODN was isolated from the blood and brain samples using DNAzol according to manufacturer's protocol with modification. Briefly, the brains (˜300 mg) were homogenized in 3 ml of DNAzol using a tissue homogenizer and the whole blood samples (100 μl) were lysed in 1 ml of DNAzol BD. After sitting 10 min at room temperature, the homogenates/lysates were subjected to two extractions using phenol/chloroform/isoamyl alcohol (25:24:1). The supernatants were transferred to new tubes and mixed with 2.5 times of ice-cold ethanol. After 1 hr incubation at −20□ followed by centrifugation (5,000 g×5 min), the resulting pellets were dissolved in 100 μl of TE buffer, applied to a 12% polyacrylamide gel (acrylamide/bis-acrylamide 19:1, 5% C) for electrophoresis (200 V). The gel was dried and exposed to phosphor imaging screen for 24 hr and the screen was then scanned using a phosphor-imager.

To determine the level of intact dsODN in blood, blood samples after administration of the targeted bioPSL(PEI2.7/³²P-dsODN) were subject to solubilization and extraction of nucleic acids. The nucleic acids were applied to 12% polyacrylamide gel for electrophoresis and then autoradiograms were obtained from the gel. The autoradiogram obtained from blood samples revealed strong bands comigrating with intact ³²P-dsODN at each sampling time point. The intensity of the intact ³²P-dsODN bands was quantified and converted to % ID/ml blood by comparison to the intensity of the control band representing 10% ID/ml blood. The blood concentration of the intact ³²P-dsODN at each time point was lower than, but comparable to the corresponding blood concentration determined by total radioactivity counting in pharmacokinetic studies. Release from particles and enzymatic degradation of ³²P-dsODN would yield a series of smaller oligonucleotides, resulting in bands with greater electrophoretic mobility. At each sampling time point, some degree of degradation was observed as shown by the bands below the intact ³²P-dsODN band.

To determine the level of intact dsODN accumulated in brain after i.v. administration of bioPSL particles, brain samples after administration of the targeted bioPSL(PEI2.7/³²P-dsODN) were subject to solubization and extraction of nucleic acids. The nucleic acids were applied to 12% polyacrylamide gel for electrophoresis and then autoradiograms were obtained from the gel. The autoradiogram obtained from brain samples also revealed single bands comigrating with intact ³²P-dsODN at each time point. The intensity of the intact ³²P-dsODN bands was quantitated and converted to % ID/g by comparison to the intensity of the control band representing 1.0% ID/g. The brain accumulation of the intact ³²P-dsODN at each time point was comparable to the corresponding brain accumulation determined by total radioactivity counting in pharmacokinetic studies. At each time point, degradation products were also detected as bands below the intact ³²P-dsODN band.

### Example 11

### Brain Uptake of bioPSL after ICA Perfusion by LSCM

Unilateral vascular brain perfusions were performed in anesthetized male Balb/c mice (Isoflurane with 0.7 L/min N₂O, 0.3 L/min O₂) via anterograde cannulation of the common carotid artery after ligation of the external carotid artery, and common carotid artery. The occipital and pterygopalatine artery were cauterized by electric coagulator. The double labeled bioPSL(TMR-PEI/A488-dsODN) particles were conjugated with 8D3SA at SA:biotin molar ratio 1:4. For control, a conjugate of non-specific isotype matched antibody UPC10 and SA was prepared and coupled to bioPSL particles. The antibody targeted bioPSL(TMR-PEI/A488-dsODN) were then diluted in Krebs-Henseleit buffer (KHB, 120 mM NaCl, 4.7 mM KCl, 25 mM NaHCO₃, 1.2 mM MgSO₄, 1.2 mM KH₂PO₄, 2.5 mM CaCl₂, 10 mM D-glucose, pH 7.3), equilibrated with 95% O₂/5% CO₂, at a concentration of 0.5 μM dsODN and then perfused at a flow rate of 250 μl/min for 10 min (15.5 μg dsODN, 1.3 mg total lipids, 290 μg protein per animal) by microsyringe pump (CMA100, Carnegie Medicine AB, Sweden) immediately after cutting the jugular vein. Immediately after the 10 min perfusion, TRIS buffered saline (TBS, 10 mM tris, pH 7.4) was perfused at a flow rate of 1 ml/min for 1 min. The brain was then fixed by perfusing 50 ml of 4% paraformaldehyde in TBS at a flow rate of 2 ml/min. The brain was removed and divided into 4 mm coronal slices. The slices were immersion-fixed with the same fixative for 2 hours. Coronal sections of 40 μm thickness were prepared in 10 mM PBS by a vibratome and collected on cover slips. After counter-staining of nuclei with DRAQ5, the sections were observed with a LSCM.

The brain endothelial uptake of the targeted 8D3SA-bioPSL(TMR-PEI2.7/A488-dsODN) particles was visualized by LSCM. After ICA perfusion of the fluorescent labeled particles and perfusion fixation with paraformaldehyde, coronal brain sections of the brain were examined. The particles could readily be visualized in brain microvasculature endothelial cells. Sections from control brain perfused with KHB buffer showed no signal. Sections from control brain perfused with non-specific isotype antibody (UPC-10) conjugated bioPSL particles showed very few particles. The uptake of the 8D3 targeted particles indicates that the uptake was specific and mediated by transferrin receptors (TfR) at brain capillary endothelial cells. Most particles had undergone endocytosis, as shown by intracellular localization in close proximity to the endothelial cell nucleus.

The bioPSL particles should remain stable in circulation and be internalized as intact liposomal particles with PEI/dsODN complexes inside. To determine whether the internalized particles retain the intrapped PEI/dsODN complexes, FRET analysis by acceptor bleaching was performed on the internalized particles. FRET efficiencies of about 30-40% were found, confirming that PEI and dsODN were still in close proximity within these internalized complexes. The presence of FRET on the internalized particles indicates that the targeted bioPSL particles are taken up as intact particles.

The internalization of the targeted bioPSL particles after ICA perfusion was further confirmed by colocalization of the particles with intracellular compartments early endosomes. The brain sections perfused with the antibody-targeted single labeled bioPSL(PEI/TMR-dsODN) particles were immunostained against EEA1 with polyclonal antibody. The immunostaining of the brain sections with EEA1 antibody showed vesicular compartments in capillary endothelial cells. The colocalization of the particles with EEA1 supports that the particles are internalized by TfR-mediated uptake.

For immunostaining of laminin-1, brain sections with 20 μm thickness were prepared as above and then blocked with 1% normal goat serum (Santa Cruz, Calif.) for 30 min. After blocking, the sections were incubated with rabbit anti-mouse laminin-1 (1.0 μg/mL) for 4 hr, followed by 4 hr incubation with secondary antibody Alexa Fluor-633 goat anti-rabbit IgG (1.0 μg/mL). After washing with PBS and counter-staining of nuclei with DRAQ5, the brain sections were mounted with glycerol and observed with LSCM. Control sections with no primary antibody treatment resulted in no staining.

For immunostaining of either EEA1 or CAV1, the single labeled bioPSL(PEI/TMR-dsODN) was prepared and administrated to mice as described above. The brain sections with 20 μm thickness were prepared as above and then blocked with 1% normal chicken serum for 30 min. After blocking, the sections were incubated with either goat anti-human polyclonal antibody (1.0 μg/mL) to early endosome antigen 1 (EEA1) or rabbit anti-human polyclonal antibody (1.0 μg/mL) to caveolin-1 (CAV1) in 10 mM PBS containing 0.05% sodium azide and 0.2% saponin for 4 hr at RT. Secondary antibodies (1 μg/mL) were Alexa Fluor-488 chicken anti-goat IgG for EEA1 and Alexa Fluor-488 chicken anti-rabbit IgG for CAV1 applied in 10 mM PBS containing 1% normal chicken serum and 0.05% sodium azide for 4 hr at RT. After washing with 10 mM PBS and counter-staining of nuclei with DRAQ5, the brain sections were mounted with glycerol mounting media and observed with LSCM. For control, the antibodies incubated with 5 times molar excess of blocking peptides were used and resulted in no staining.

The brain section after ICA perfusion of antibody-targeted single labeled bioPSL(PEI/TMR-dsODN) particles were immunostained against CAV1 with polyclonal antibody. The immunostaining of the brain sections with CAV1 antibody showed membrane and cytoplasmic staining of capillary endothelial cells. The single-labeled particles were found with CAV1 staining. The colocalization of the particles with CAV1 confirms that the particles are internalized.

To determine whether the particles can reach the brain parenchyma beyond the BBB, the brain sections obtained after ICA perfusion of antibody-targeted, double-labeled bioPSL(TMR-PEI/A488-dsODN) particles were immunostained against laminin-1. Laminins are one of the major structural components of the extracellular basement membrane, which forms a continuous sleeve around the basal surface of endothelial capillary tubes and plays an important role in the maintenance of vessel wall integrity. Laminin-1 (LAM1) is an isoform detected in blood vessel in the central nervous system (CNS) (Hallmann, Horn et al. 2005).

After uptake and passage across the brain endothelial cell monolayer, the particles still face the endothelial cell basement membrane and parenchymal basement membrane as obstacles before they can reach the brain parenchyma. Immunostaining of the brain sections with LAM1 antibody revealed basement membranes around brain capillary endothelial cells. The particles found in perivascular space between endothelial basement membrane and parenchymal basement membrane is evidence that the particles can reach the brain parenchyma beyond the BBB.

### Example 12

### Brain Uptake and Organ Distribution after I.V. Bolus by LSCM

The antibody targeted bioPSL(TMR-PEI/A488-dsODN) was prepared as described above at a concentration of 1.5 μM dsODN and injected via the jugular vein by i.v. bolus (100 μl, 1.9 μg dsODN, 0.16 mg total lipids, 35 μg protein per animal). After 30 minutes, the animal was sacrified by transcardial perfusion of 5 ml of ice-cold TBS, followed by 50 ml of 4% paraformaldehyde in TBS at a flow rate of 4 ml/min. The brain was isolated and divided into 4 mm coronal slices. The slices were immersion-fixed with the same fixative for 2 hours. The coronal sections of 40 μm thickness were prepared in 10 mM PBS by a vibratome and collected on cover slips. After counter-staining of nuclei with DRAQ5, the sections were observed with a LSCM. Other major organs such as lung, heart, liver, spleen, and kidney were also treated as with brain.

The brain sections after i.v. administration of the targeted bioPSL(TMR-PEI/A488-dsODN) particles were examined under LSCM. The particles are readily found in brain endothelial cells. Endothelial uptake of the particles was confirmed by the localization of the particles at the abluminal side of the endothelial nucleus.

To determine whether the internalized particles retain the intrapped PEI/dsODN complexes, FRET analysis by acceptor bleaching was performed on the internalized particles.

FRET efficiencies of about 30˜40% were found, confirming that PEI and dsODN were still in close proximity within these internalized complexes. The presence of FRET on the internalized particles indicates that the targeted bioPSL particles remain stable in blood circulation and taken up as intact particles.

Other major organs such as lung, heart, liver, spleen and kidney, were also examined under LSCM. The particles were found in liver and spleen at very high levels while accumulation in heart, lung and kidney was insignificant.

The almost exclusive accumulation of the targeted bioPSL particles in liver and spleen indicates that the RES is a major player in clearance of the particles in circulation. The very low level of accumulation in lung indicates that the particles do not aggregate in blood circulation, otherwise significant accumulation would be observed.

### Example 13

### In Vivo Brain Uptake of Targeted bPP/dsODN Complexes by LSCM

The block copolymer biotin-PEG(3700)-PEI(2700) (bPP) was labeled with TMR (TMR-bPP) as described for PEI. Double labeled complexes (TMR-bPP/FL-dsODN) were prepared with FL-dsODN and TMR-bPP at N/P ratio=6. The desired amounts of dsODN and polymer were diluted separately in PBS to a final volume of 500 μl. After 10 min incubation at room temperature, the polymer solutions were then transferred to the dsODN solution by fast addition and vortexed immediately. After additional 10 min incubation at RT, the complexes were conjugated with 8D3SA at a biotin:SA molar ratio 26 and then diluted with KHB to a concentration of 1.0 μM dsODN. The antibody-targeted bPP/dsODN complexes were perfused via ICA as described below.

For i.v. infusion, the complexes were prepared as described above at a concentration of 5 μM dsODN and then infused via cannulation of the jugular vein at an infusion rate of 50 μl/min for 10 min. At 20 min after the end of the infusion, 50 ml of fixative (4% PFA) was perfused by transcardial perfusion at a flow rate of 4 ml/min. For i.v. bolus, the complexes were prepared as described above at a concentration of 25 μM dsODN and then 100 μl of the complexes was injected via the jugular vein. After 30 min, 50 ml of the same fixative was perfused by transcardial perfusion at a flow rate of 4 ml/min. The brains and other major organs were removed and treated as described below. The coronal sections of 40 μm thickness were prepared with a vibratome in 10 mM PBS and collected on cover slips. After counter-staining of nuclei with DRAQ5, the sections were observed with a LSCM.

The biotinylated PEG-grafted PEI (bPP) has been extensively studied in our laboratory. In vitro and in vivo studies with the bPP concluded that the bPP/dsODN complexes should undergo significant uptake by brain endothelial cells. The brain endothelial uptake of the bPP/dsODN complexes was investigated by LSCM in comparison to the bioPSL particles.

After ICA perfusion of the fluorescent labeled particles, the particles could readily be visualized in brain microvasculature endothelial cells. Most particles had undergone endocytosis, as shown by intracellular localization in close proximity to the endothelial cell nucleus.

In addition, FRET analysis with acceptor bleaching was performed on internalized particles. FRET efficiencies of about 40% were found, confirming that bPP and dsODN were still in close proximity within these internalized complexes. The bPP/dsODN complexes were colocalized with EEA1 or CAV1, supporting that the complexes are internalized by the endothelial cells.

Morever, the complexes could readily be visualized in brain microvasculature endothelial cells after i.v. administration of the fluorescent labeled particles. Most particles had undergone endocytosis, as shown by intracellular localization in close proximity to the endothelial cell nucleus. In addition, FRET analysis with acceptor bleaching was performed on internalized particles. FRET efficiencies of about 50% were found, confirming that bPP and dsODN were still in close proximity within these internalized complexes. Other major organs such as lung, heart, liver, spleen and kidney, were also examined under LSCM. The particles were found in liver and spleen at very high levels with mild accumulation in lung and kidney.

### REFERENCES CITED

The following references, to the extent that they provide exemplary procedural or other details supplementary to those set forth herein, are specifically incorporated herein by reference.

### U.S. Patent Documents

U.S. Pat. No. 6,120,798 issued on Sep. 19, 2000 to Allen et al.
U.S. Pat. No. 6,056,973 issued on May 2, 2000 to Allen et al.
U.S. Pat. No. 6,316,024 issued on Nov. 13, 2001 to Allen et al.
U.S. Pat. No. 6,372,250 B1 issued on Apr. 16, 2002 to Pardridge et al.
U.S. Patent Publication 2005/0042298 A1 published on Feb. 24, 2005 with Pardridge et al. listed as inventors.
U.S. Patent Publication 2005/0202075 A1 published on Sep. 15, 2005 with Pardridge et al. listed as inventors.
U.S. Patent Publication 2005/0152963 A1 published on Jul. 14, 2005 with Huwyler et al. listed as inventors.
U.S. Patent Publication 2005/0053590 A1 published on Mar. 10, 2005, with Meininger, C. J., listed as the inventor.
U.S. Patent Publication 2007/0110798 A1 published on May 17, 2007, with Drummond et al. listed as inventors.

### Foreign Patent Documents

German Offenlegungsshrift DE 197 43 135 A1 (INID# 10), published on Apr., 1, 1999 (INID #43), with Hoechst Marion Roussel Deutschland GmbH listed as the Applicant (INID #77).

### References

Szebeni, J. (1998). “The interaction of liposomes with the complement system.” Crit. Rev Ther Drug Carrier Syst 15(1): 57-88.
Abuchowski, A., J. R. McCoy, N. C. Palczuk, T. van Es and F. F. Davis (1977). “Effect of covalent attachment of polyethylene glycol on immunogenicity and circulating life of bovine liver catalase.” J Biol Chem 252(11): 3582-6.
Harris, J. M., N. E. Martin and M. Modi (2001). “Pegylation: a novel process for modifying pharmacokinetics.” Clin Pharmacokinet 40(7): 539-51.
Petersen, H., P. M. Fechner, A. L. Martin, K. Kunath, S. Stolnik, C. J. Roberts, D. Fischer, M. C. Davies and T. Kissel (2002). “Polyethylenimine-graft-poly(ethylene glycol) copolymers: influence of copolymer block structure on DNA complexation and biological activities as gene delivery system.” Bioconjug Chem 13(4): 845-54.
Blume, G., G. Cevc, M. D. Crommelin, I. A. Bakker-Woudenberg, C. Kluft and G. Storm (1993). “Specific targeting with poly(ethylene glycol)-modified liposomes: coupling of homing devices to the ends of the polymeric chains combines effective target binding with long circulation times.” Biochim Biophys Acta 1149(1): 180-4.
Allen, T. M., C. Hansen, F. Martin, C. Redemann and A. Yau-Young (1991). “Liposomes containing synthetic lipid derivatives of poly(ethylene glycol) show prolonged circulation half-lives in vivo.” Biochim Biophys Acta 1066(1): 29-36.
Papahadjopoulos, D., T. M. Allen, A. Gabizon, E. Mayhew, K. Matthay, S. K. Huang, K. D. Lee, M. C. Woodle, D. D. Lasic, C. Redemann and et al. (1991). “Sterically stabilized liposomes: improvements in pharmacokinetics and antitumor therapeutic efficacy.” Proc Natl Acad Sci USA 88(24): 11460-4.
Mori, A., A. L. Klibanov, V. P. Torchilin and L. Huang (1991). “Influence of the steric barrier activity of amphipathic poly(ethyleneglycol) and ganglioside GM1 on the circulation time of liposomes and on the target binding of immunoliposomes in vivo.” FEBS Lett 284(2): 263-6.
Senior, J., C. Delgado, D. Fisher, C. Tilcock and G. Gregoriadis (1991). “Influence of surface hydrophilicity of liposomes on their interaction with plasma protein and clearance from the circulation: studies with poly(ethylene glycol)-coated vesicles.” Biochim Biophys Acta 1062(1): 77-82.
Uster, P. S., T. M. Allen, B. E. Daniel, C. J. Mendez, M. S, Newman and G. Z. Zhu (1996). “Insertion of poly(ethylene glycol) derivatized phospholipid into pre-formed liposomes results in prolonged in vivo circulation time.” FEBS Lett 386(2-3): 243-6.
Woodle, M. C. (1998). “Controlling liposome blood clearance by surface-grafted polymers.” Adv Drug Deliv Rev 32(1-2): 139-152.
Kakudo, T., S. Chaki, S. Futaki, I. Nakase, K. Akaji, T. Kawakami, K. Maruyama, H. Kamiya and H. Harashima (2004). “Transferrin-modified liposomes equipped with a pH-sensitive fusogenic peptide: an artificial viral-like delivery system.” Biochemistry 43(19): 5618-28.
Silvius, J. R. and M. J. Zuckermann (1993). “Interbilayer transfer of phospholipid-anchored macromolecules via monomer diffusion.” Biochemistry 32(12): 3153-61.
Bedu-Addo, F. K., P. Tang, Y. Xu and L. Huang (1996). “Effects of polyethyleneglycol chain length and phospholipid acyl chain composition on the interaction of polyethyleneglycol-phospholipid conjugates with phospholipid: implications in liposomal drug delivery.” Pharm Res 13(5): 710-7.
Litzinger, D. C., A. M. Buiting, N. van Rooijen and L. Huang (1994). “Effect of liposome size on the circulation time and intraorgan distribution of amphipathic poly(ethylene glycol)-containing liposomes.” Biochim Biophys Acta 1190(1): 99-107.
Braet, F., R. De Zanger, M. Baekeland, E. Crabbe, P. Van Der Smissen and E. Wisse (1995). “Structure and dynamics of the fenestrae-associated cytoskeleton of rat liver sinusoidal endothelial cells.” Hepatology 21(1): 180-9.
Moghimi, S. M., C. J. Porter, I. S. Muir, L. Illum and S. S. Davis (1991). “Non-phagocytic uptake of intravenously injected microspheres in rat spleen: influence of particle size and hydrophilic coating.” Biochem Biophys Res Commun 177(2): 861-6.
Allen, T. M. and C. Hansen (1991). “Pharmacokinetics of stealth versus conventional liposomes: effect of dose.” Biochim Biophys Acta 1068(2): 133-41.
Laverman, P., O. C. Boerman, W. J. G. Oyen, F. H. M. Corstens and G. Storm (2001). “In vivo applications of PEG liposomes: unexpected observations.” Crit Rev Ther Drug Carrier Syst 18(6): 551-66.
Coukell, A. J. and C. M. Spencer (1997). “Polyethylene glycol-liposomal doxorubicin. A review of its pharmacodynamic and pharmacokinetic properties, and therapeutic efficacy in the management of AIDS-related Kaposi's sarcoma.” Drugs 53(3): 520-38.
Kakudo, T., S. Chaki, S. Futaki, I. Nakase, K. Akaji, T. Kawakami, K. Maruyama, H. Kamiya and H. Harashima (2004). “Transferrin-modified liposomes equipped with a pH-sensitive fusogenic peptide: an artificial viral-like delivery system.” Biochemistry 43(19): 5618-28.
Lopes de Menezes, D. E., L. M. Pilarski and T. M. Allen (1998). “In vitro and in vivo targeting of immunoliposomal doxorubicin to human B-cell lymphoma.” Cancer Res 58(15): 3320-30.
Harris, J. M. and R. B. Chess (2003). “Effect of pegylation on pharmaceuticals.” Nat Rev Drug Discov 2(3): 214-21.
Moghimi, S. M. and A. C. Hunter (2001). “Capture of stealth nanoparticles by the body's defences.” Crit Rev Ther Drug Carrier Syst 18(6): 527-50.
Wheeler, J. J., L. Palmer, M. Ossanlou, I. MacLachlan, R. W. Graham, Y. P. Zhang, M. J. Hope, P. Scherrer and P. R. Cullis (1999). “Stabilized plasmid-lipid particles: construction and characterization.” Gene Ther 6(2): 271-81.
Stuart, D. D., G. Y. Kao and T. M. Allen (2000). “A novel, long-circulating, and functional liposomal formulation of antisense oligodeoxynucleotides targeted against MDR1.” Cancer Gene Ther 7(3): 466-75.
Lee, R. J. and L. Huang (1996). “Folate-targeted, Anionic Liposome-entrapped Polylysine-condensed DNA for Tumor Cell-specific Gene Transfer.” Journal of Biological Chemistry 271(14): 8481-8487.
Muller, K., T. Nahde, A. Fahr, R. Muller and S. Brusselbach (2001). “Highly efficient transduction of endothelial cells by targeted artificial virus-like particles.” Cancer Gene Therapy 8(2): 107-117.
Boussif, O., F. Lezoualc'h, M. A. Zanta, M. D. Mergny, D. Scherman, B. Demeneix and J. P. Behr (1995). “A versatile vector for gene and oligonucleotide transfer into cells in culture and in vivo: polyethylenimine.” Proc Natl Acad Sci USA 92(16): 7297-301.
Akinc, A., M. Thomas, A. M. Klibanov and R. Langer (2005). “Exploring polyethylenimine-mediated DNA transfection and the proton sponge hypothesis.” J Gene Med 7(5): 657-63.
Kircheis, R., L. Wightman and E. Wagner (2001). “Design and gene delivery activity of modified polyethylenimines.” Adv Drug Deliv Rev 53(3): 341-58.
Vinogradov, S. V., E. V. Batrakova and A. V. Kabanov (2004). “Nanogels for oligonucleotide delivery to the brain.” Bioconjug Chem 15(1): 50-60.
Huang, J., J. T. Buboltz and G. W. Feigenson (1999). “Maximum solubility of cholesterol in phosphatidylcholine and phosphatidylethanolamine bilayers.” Biochim Biophys Acta 1417(1): 89-100.
Stuart, D. D. and T. M. Allen (2000). “A new liposomal formulation for antisense oligodeoxynucleotides with small size, high incorporation efficiency and good stability.” Biochim Biophys Acta 1463(2): 219-29.
Van Rompaey, E., Y. Engelborghs, N. Sanders, D. S. S C and J. Demeester (2001). “Interaction between oligonucleotides and cationic polymers investigated by fluorescence correlation spectroscopy.” Pharmaceutical Research 18(7): 928-36.
Itaka, K., A. Harada, K. Nakamura, H. Kawaguchi and K. Kataoka (2002). “Evaluation by fluorescence resonance energy transfer of the stability of nonviral gene delivery vectors under physiological conditions.” Biomacromolecules 3(4): 841-5.
Parker, A., K. Miles, K. H. Cheng and J. Huang (2004). “Lateral distribution of cholesterol in dioleoylphosphatidylcholine lipid bilayers: cholesterol-phospholipid interactions at high cholesterol limit.” Biophys J 86(3): 1532-44.
Edelman, L. M., R. Cheong and J. D. Kahn (2003). “Fluorescence resonance energy transfer over approximately 130 basepairs in hyperstable lac repressor-DNA loops.” Biophys J 84(2 Pt 1): 1131-45.
von Harpe, A., H. Petersen, Y. Li and T. Kissel (2000). “Characterization of commercially available and synthesized polyethylenimines for gene delivery.” J Control Release 69(2): 309-22.
Shinitzky, M. and Y. Barenholz (1978). “Fluidity parameters of lipid regions determined by fluorescence polarization.” Biochimica et biophysica Acta 515(4): 367-394.
Parker, A., K. Miles, K. H. Cheng and J. Huang (2004). “Lateral distribution of cholesterol in dioleoylphosphatidylcholine lipid bilayers: cholesterol-phospholipid interactions at high cholesterol limit.” Biophys J 86(3): 1532-44.
Kakehi, K., Y. Oda and M. Kinoshita (2001). “Fluorescence polarization: analysis of carbohydrate-protein interaction.” Anal Biochem 297(2): 111-6.
Clamme, J. P., J. Azoulay and Y. Mely (2003). “Monitoring of the formation and dissociation of polyethylenimine/DNA complexes by two photon fluorescence correlation spectroscopy.” Biophys J 84(3): 1960-8.
Linnertz, H., P. Urbanova and E. Amler (1997). “Quenching of 7-chloro-4-nitrobenzo-2-oxa-1,3-diazole-modified Na+/K+-ATPase reveals a higher accessibility of the low-affinity ATP-binding site.” FEBS Lett 419(2-3): 227-30.
Cameron, P. L., J. W. Ruffin, R. Bollag, H. Rasmussen and R. S. Cameron (1997). “Identification of caveolin and caveolin-related proteins in the brain.” J Neurosci 17(24): 9520-35.
Fischer, D., R. Bhattacharya, B. Osburg and U. Bickel (2005). “Inhibition of monocyte adhesion on brain-derived endothelial cells by NF-kappaB decoy/polyethylenimine complexes.” J Gene Med.
Fischer, D., B. Osburg, H. Petersen, T. Kissel and U. Bickel (2004). “Effect of poly(ethylene imine) molecular weight and pegylation on organ distribution and pharmacokinetics of polyplexes with oligodeoxynucleotides in mice.” Drug Metab Dispos 32(9): 983-92.
Hallmann, R., N. Horn, M. Selg, O. Wendler, F. Pausch and L. M. Sorokin (2005). “Expression and function of laminins in the embryonic and mature vasculature.” Physiol Rev 85(3): 979-1000.
Kichler, A., C. Leborgne, E. Coeytaux, O. Danos (2001) “Polyethylenimine-Mediated Gene Delivery: a Mechanistic Study.” J Gene Med 3(2):135-44.
Thomas, M., A. M. Klibanov (2002) “Enhancing Polyethylenimine's Delivery of Plasmid DNA into Mammalian Cells.” Proc Nat Acad Sci 99(23):14640-14645.

What is claimed:

1. A liposome-encapsulated nucleic acid/polymer complex comprising
a nucleic acid;
a polycationic polymer;
a phospholipid;
wherein the nucleic acid is complexed with the polycationic polymer to form a nucleic acid/polymer complex, and the nucleic acid/polymer complex is encapsulated in a liposome comprising the phospholipid.

2. The liposome-encapsulated nucleic acid/polymer complex of claim 1 wherein the nucleic acid is a therapeutic agent.

3. The liposome-encapsulated nucleic acid/polymer complex of claim 1 wherein the nucleic acid is a therapeutic agent capable of modulating signaling pathways in endothelial cells.

4. The liposome-encapsulated nucleic acid/polymer complex of claim 1, wherein the liposome comprises a targeting molecule.

5. The liposome-encapsulated nucleic acid/polymer complex of claim 4, wherein the targeting molecule is biotin, an antibody, a ligand, or a small molecule.

6. A method for delivering a therapeutic agent to an organism comprising the step of
administering the liposome-encapsulated nucleic acid/polymer complex of claim 1 to the organism.

7. A method for delivering a therapeutic agent across the blood brain barrier of an organism comprising the step of administering the liposome-encapsulated nucleic acid/polymer complex of claim 1 to the organism.

8. A liposome-encapsulated nucleic acid/polymer complex comprising
a nucleic acid;
polyethylenimine;
a phospholipid;
wherein the nucleic acid is complexed with the polyethylenimine to form a nucleic acid/polymer complex, and the nucleic acid/polymer complex is encapsulated in a liposome comprising the phospholipid.

9. The liposome-encapsulated nucleic acid/polymer complex of claim 8 wherein the nucleic acid is a therapeutic agent.

10. The liposome-encapsulated nucleic acid/polymer complex of claim 8 wherein the nucleic acid is a therapeutic agent capable of modulating signaling pathways in endothelial cells.

11. The liposome-encapsulated nucleic acid/polymer complex of claim 8, wherein the liposome comprises a targeting molecule.

12. The liposome-encapsulated nucleic acid/polymer complex of claim 11, wherein the targeting molecule is biotin, an antibody, a ligand, or a small molecule.

13. A method for delivering a therapeutic agent to an organism comprising the step of
administering the liposome-encapsulated nucleic acid/polymer complex of claim 8 to the organism.

14. A method for delivering a therapeutic agent across the blood brain barrier of an organism comprising the step of
administering the liposome-encapsulated nucleic acid/polymer complex of claim 8 to the organism.

15. A liposome-encapsulated nucleic acid/polymer complex comprising
a oligodeoxynucleotide;
a polycationic polymer;
a phospholipid;
wherein the double stranded oligodeoxynucleotide is complexed with the polycationic polymer to form a nucleic acid/polymer complex, and the nucleic acid/polymer complex is encapsulated in a liposome comprising the lipid.

16. The liposome-encapsulated nucleic acid/polymer complex of claim 15 wherein the oligodeoxynucleotide is a therapeutic agent.

17. The liposome-encapsulated nucleic acid/polymer complex of claim 15 wherein the oligodeoxynucleotide is a therapeutic agent capable of modulating signaling pathways in endothelial cells.

18. The liposome-encapsulated nucleic acid/polymer complex of claim 15, wherein the liposome comprises a targeting molecule.

19. The liposome-encapsulated nucleic acid/polymer complex of claim 18, wherein the targeting molecule is biotin, an antibody, a ligand, or a small molecule.

20. A method for delivering a therapeutic agent to an organism comprising the step of
administering the liposome-encapsulated nucleic acid/polymer complex of claim 15 to the organism.

21. A method for delivering a therapeutic agent across the blood brain barrier of an organism comprising the step of
administering the liposome-encapsulated nucleic acid/polymer complex of claim 15 to the organism.

22. A method for producing a liposome-encapsulated nucleic acid/polymer complex, comprising the steps of
adding a polycationic polymer solution to an oligodeoxynucleotide solution at a polycationic polymer to oligodeoxynucleotide ratio of about 5 to 7 to form a nucleic acid/polymer solution;
preparing multilamellar anionic liposomes;
extruding the multilamellar anionic liposomes to form unilamellar liposomes; and
mixing the unilamellar liposomes with the nucleic acid/polymer solution to form a liposome solution.

23. The method of claim 22, further comprising an extrusion step comprising extruding the liposome mixture through a membrane, wherein the membrane allows liposomes of about 80 nm to 180 nm in diameter to be extruded.

24. A method for producing a liposome-encapsulated nucleic acid/polymer complex, comprising the steps of
adding a polycationic polymer solution to an oligodeoxynucleotide solution at a polycationic polymer to oligodeoxynucleotide ratio of about 5 to 7 to form a nucleic acid/polymer solution;
diluting a phospholipid in chloroform and adding MeOH and the nucleic acid/polymer solution to form a lipid/nucleic acid/polymer solution;
incubating the lipid/nucleic acid/polymer solution at room temperature;
centrifuging the lipid/nucleic acid/polymer solution;
removing the aqueous phase from the lipid/nucleic acid/polymer solution to form a liposome mixture;
adding a lipid mixture, the lipid mixture comprising phospholipid, to the liposome mixture followed by mixing;
placing the liposome mixture under a vacuum for a period of time.

25. The method of claim 24, further comprising an extrusion step comprising extruding the liposome mixture through a membrane, wherein the membrane allows liposomes of about 80 nm to 180 nm in diameter to be extruded.

26. A method for producing a liposome-encapsulated nucleic acid/polymer complex, comprising the steps of
adding a polycationic polymer solution to an oligodeoxynucleotide solution at a polycationic polymer to oligodeoxynucleotide ratio of about 5 to 7 to form a nucleic acid/polymer solution;
diluting a phospholipid in chloroform, followed by removing the chloroform to form a dried phospholipid;
adding the nucleic acid/polymer complex to the dried phospholipid to form a liposome solution;
incubating the liposome solution at room temperature with mixing.

27. The method of claim 26, further comprising an extrusion step comprising extruding the liposome mixture through a membrane, wherein the membrane allows liposomes of about 80 nm to 180 nm in diameter to be extruded.
